(19) United States
(12) Patent Application Publication
(10) Pub. No.: US 2004/0068217 A1
(43) Pub. Date: Apr. 8, 2004

(54) Self-expanding exterior connectors for creating anastomoses to small-diameter vessels and methods of use

(75) Inventors: Paul J. Hindrichs, Plymouth, MN (US); Michael P. Brenzel, St. Paul, MN (US); Richard G. Cornelius, Wayzata, MN (US); William J. Swanson, St. Paul, MN (US)

Correspondence Address: FISH & NEAVE, 1251 AVENUE OF THE AMERICAS, 50TH FLOOR, NEW YORK, NY, 10020-1105, US

(73) Assignee: St. Jude Medical ATG, Inc.

(21) Appl. No.: 10/678,403

(22) Filed: Oct. 3, 2003

Related U.S. Application Data
(60) Provisional application No. 60/416,485, filed on Oct. 4, 2002.

(51) Int. Cl. A61F 13/00

(52) U.S. Cl. 602/41

(57) ABSTRACT

Methods and apparatus for making an anastomotic connection between a first aperture in a side wall of a graft conduit and a second aperture in a side wall of a body tissue conduit using a hollow self-expanding exterior connector are provided. The tissue about the first aperture is introduced into the hollow connector and is retained by first members of a distal portion of the connector. A delivery tool then collapses the distal perimeter defined by the first members by deforming the connector and delivers the first members into the lumen of the body tissue conduit via the second aperture. Upon inserting the first members into the body tissue conduit, the tool is disemployed and the connector reforms such that the first members and a more proximal portion of the connector external to the body tissue conduit press together the walls of the two conduits around the apertures.

This application claims the benefit of U.S. provisional patent application No. 60/416,485, filed Oct. 4, 2002, which is hereby incorporated by reference herein in its entirety.

### BACKGROUND OF THE INVENTION

This invention relates to medical grafting apparatus and methods for creating anastomoses and, more particularly, to apparatus and methods for creating anastomoses to small diameter body fluid conduits in a patient.

There are many medical procedures in which it is necessary to make an anastomotic connection between two tubular body fluid conduits in a patient. An anastomotic connection (or anastomosis) is a connection which allows body fluid flow between the lumens of the two conduits that are connected, preferably without allowing body fluid to leak out of the conduits at the location of the connection. As just one example of a procedure in which an anastomosis is needed, in order to bypass an obstruction in a patient's coronary artery, a tubular graft supplied with blood may be connected via an anastomosis to the coronary artery downstream from the obstruction. The anastomosis may be between the end of the graft and an aperture in the side wall of the coronary artery (a so-called end-to-side anastomosis), or the anastomosis may be between an aperture in the side wall of the graft and an aperture in the side wall of the coronary artery (a so-called side-to-side anastomosis). The graft may be natural conduit, synthetic conduit, or a combination of natural and synthetic conduits. If natural conduit is used, it may be wholly or partly relocated from elsewhere in the patient (e.g., wholly relocated saphenous vein graft (“SVG”) or partly relocated internal mammary artery (“IMA”)). Alternatively, no relocation of the graft may be needed (e.g., a length of vein on the heart becomes a “graft” around an obstruction in an immediately adjacent coronary artery). More than one anastomosis may be needed. For example, a second anastomosis may be needed between an upstream portion of the graft conduit and the aorta or the coronary artery upstream from the obstruction in that artery. Again, this second anastomosis may be either an end-to-side anastomosis or a side-to-side anastomosis. Alternatively, no second upstream anastomosis may be required at all (e.g., if the graft is an only-partly-relocated IMA).

In the case of making a conventional end-to-side anastomosis between a vein graft and the coronary artery, certain difficulties may arise. First, the relative sizes of the coronary artery and the vein graft are different. For example, the coronary artery may typically have an inner diameter of about 1.0 to 3.0 mm, whereas a vein graft, such as the saphenous vein, may typically have an inner diameter of about 4.0 to 8.0 mm. This discrepancy between vessel diameters, i.e., a “caliber mismatch,” may present a challenge to the physician to match the end of the relatively larger vein graft to an aperture in the side wall of the relatively smaller coronary artery. The resulting quality and amount of flow between the vein graft and the coronary artery, along with the provision of an effective hemodynamic seal between the two conduits, is often dependent upon the physician's skill in making an effective junction between the two conduits.

Second, conventional end-to-side anastomosis typically joins the graft conduit to the coronary artery at an angle with respect to the lumen of the coronary artery, thus forming a junction at the wall of the coronary artery. Further away from this junction, the vein graft tends to lie against the heart structure, or substantially parallel to the lumen of the coronary artery. The transition of the vein graft from a substantially perpendicular juncture to the coronary artery to a substantially parallel position with respect to the coronary artery wall often occurs abruptly, which may result in kinking of the vein graft, with possibly reduced blood flow.

Third, joining vessels having relatively small diameters (e.g., 1-4 millimeters) presents the additional consideration of keeping the vessels open after the anastomosis has been made. Currently, the ostium diameter of anastomosis utilizing commercially available connectors at the proximal anastomosis between the graft and the aorta is limited by and usually smaller than the diameter of the graft. It is therefore helpful to provide the anastomosis with a diameter equal to or larger than the diameter of the smaller vessel being joined in order to minimize the risk of closing off the flow due to the natural healing response.

Accordingly, it would be desirable to provide apparatus for making a side-to-side anastomosis whose ostium diameter is larger than that of the graft.

It is another object of the invention to provide apparatus for making a side-to-side anastomosis whose ostium diameter is larger than that of the graft by using an exterior connector for minimizing the surface area of connector material in the blood flow path to reduce clotting and foreign body response, for example.

### SUMMARY OF THE INVENTION

It is therefore an object of this invention to provide apparatus for making a side-to-side anastomosis whose ostium diameter is larger than that of the graft.

It is therefore also an object of this invention to provide apparatus for making a side-to-side anastomosis whose ostium diameter is larger than that of the graft by using an exterior connector for minimizing the surface area of connector material in the blood flow path.

In accordance with the present invention, an apparatus including at least a first connector is provided to create a hollow anastomotic connection between tubular body fluid conduits in a patient. A particular application of the invention is to join a graft conduit to a body tissue conduit in a patient in a side-to-side anastomosis whose ostium is larger than that of the graft conduit. In a first embodiment of the present invention, the first connector has a first set of members at its distal end that engage both a first conduit (e.g., the graft conduit) and a second conduit (e.g., the body tissue conduit), and a second set of members at its proximal end that contact the second conduit and press it towards the first conduit.

The first connector self-expands to a significant extent at the distal end thereof to create an expanded and preferably non-round distal perimeter defined by the first set of members. Tissue about a first aperture in the first conduit is passed through the hollow of the first connector and engaged with the first set of members.

A delivery tool deforms the loaded connector axially and/or trans-axially to create a collapsed distal perimeter defined by the first set of members such that the first set of members are positioned to prevent trauma to the second conduit when the first connector is introduced therein. The delivery tool has a structure which may change configuration to expose the first set of members after insertion into the second conduit and to allow expansion of the distal end of the first connector therein.

A method for creating the anastomosis may include introducing the first aperture in the first conduit through the hollow of the first connector and then over its distal end to engage the first conduit with the first set of members of the first connector The aperture in the first conduit may be made in the side wall of the first conduit proximal to the distal end of the first conduit.

At the operative site, a second aperture may be made in the side wall of the second conduit. The distal end of the first connector may be deformed and the first and second apertures may be approximated so that the first set of members loaded with the first connector extends into the second conduit via the second aperture.

The first connector may reform so that it presses together the interior walls of the first and second conduits annularly around the first and second apertures. The distal end of the first connector may deform and expand axially and trans-axially. A selected delivery tool may be used to both deform the first connector and approximate the first and second apertures.

The method may also include introducing a second connector into the second conduit to hold open the second aperture and to hold together the wall tissue of the second conduit around the second aperture. The second connector may further prevent trauma to the second conduit when the first connector is introduced therein and may engage with the first connector to guarantee perimeter matching, for example.

It should be noted that the terms vessel and conduit are used interchangeable herein.

### BRIEF DESCRIPTION OF THE DRAWINGS

The above and other advantages of the invention will be made more apparent upon consideration of the following detailed description, taken in conjunction with the accompanying drawings, in which like reference characters refer to like parts throughout, and in which.

FIG. 1A is a perspective view of a vessel incision tool in accordance with the present invention;

FIG. 1B is a sectional view illustrating an early stage of a procedure involving the tool of FIG. 1A in accordance with the present invention;

FIG. 1C is a view similar to FIG. 1B illustrating a further stage of a procedure in accordance with the present invention;

FIG. 1D is a view similar to FIG. 1C illustrating a later stage of a procedure in accordance with the present invention;

FIG. 1E is a perspective view illustrating a controlled-length incision created in a vessel using the procedure of FIGS. 1B-1D in accordance with the present invention;

FIG. 1F is a perspective view of another vessel incision tool in accordance with the present invention;

FIG. 1G is another perspective view of the vessel incision tool of FIG. 1F in accordance with the present invention;

FIG. 2 is a planar development of the structure of an illustrative embodiment of a first connector constructed in accordance with the present invention;

FIG. 3 is a perspective view of the connector of FIG. 2;

FIG. 4 is a perspective view similar to FIG. 3 of the connector of FIG. 2 in another configuration;

FIG. 5 is a perspective view of a graft tissue conduit for use in a patient with a connector in accordance with the present invention;

FIG. 6 is a perspective view similar to FIG. 4 of the connector of FIG. 2 in the configuration of FIG. 4, illustrated with a sectional view of the graft tissue conduit of FIG. 5, in an early stage of a procedure in accordance with the present invention;

FIG. 7 is a simplified sectional view of the connector of FIG. 2 in the configuration of FIG. 4, illustrated with the graft tissue conduit of FIG. 5, in a further stage of a procedure in accordance with the present invention;

FIG. 8 is a view similar to FIG. 7 of the connector of FIG. 2 in yet another configuration, illustrated with the graft tissue conduit of FIG. 5, in a later stage of a procedure in accordance with the present invention;

FIG. 9 is a top elevational view of the connector of FIG. 2 in the configuration of FIG. 8, illustrated with the graft tissue conduit of FIG. 5, in the later stage of a procedure of FIG. 8, but with a portion of the graft tissue conduit omitted;

FIG. 10 is a planar development of the structure of another illustrative embodiment of a first connector constructed according to the present invention;

FIG. 11 is a perspective view of the connector of FIG. 10;

FIG. 12 is a perspective view similar to FIG. 11 of the connector of FIG. 10 in another configuration;

FIG. 13 is a perspective view similar to FIG. 12 of the connector of FIG. 10 in the configuration of FIG. 12, illustrated with the graft tissue conduit of FIG. 5, in an early stage of a procedure in accordance with the present invention;

FIG. 14 is a simplified sectional view of the connector of FIG. 10 in the configuration of FIG. 12, illustrated with the graft tissue conduit of FIG. 5, in a further stage of a procedure in accordance with the present invention;

FIG. 15 is a view similar to FIG. 13 of the connector of FIG. 10 in yet another configuration, illustrated with the graft tissue conduit of FIG. 5, in a later stage of a procedure in accordance with the present invention;

FIG. 16 is a top elevational view of the connector of FIG. 10 in the configuration of FIG. 15, illustrated with the graft tissue conduit of FIG. 5, in the later stage of a procedure of FIG. 15, but with a portion of the graft tissue conduit omitted;

FIG. 17 is a planar development of the structure of yet another illustrative embodiment of a first connector constructed in accordance with the present invention;

FIG. 18 is a perspective view of the connector of FIG. 17, illustrated with the graft tissue conduit of FIG. 5, in an early stage of a procedure in accordance with the present invention;

FIG. 19 is a simplified sectional view of the connector of FIG. 17 in another configuration, illustrated with the graft tissue conduit of FIG. 5, in a later stage of a procedure in accordance with the present invention;

FIG. 20 is a top elevational view of the connector of FIG. 17 in the configuration of FIG. 19, illustrated with the graft tissue conduit of FIG. 5, in the later stage of a procedure of FIG. 19, but with a portion of the graft tissue conduit omitted;

FIG. 21 is a side elevational view of illustrative apparatus for use in delivering and deploying a first connector in accordance with the present invention;

FIG. 22 is a front elevational view of the apparatus of FIG. 21, taken from line 22-22 of FIG. 21;

FIG. 23 is a simplified sectional view of the connector of FIG. 2 in still yet another configuration, illustrated with the graft tissue conduit of FIG. 5, the apparatus of FIG. 21, and a second connector connected to a body tissue conduit in the patient, in a still later stage of a procedure in accordance with the present invention;

FIG. 24 is a simplified sectional view of the connector of FIG. 10 in still yet another configuration, illustrated with the graft tissue conduit of FIG. 5, the apparatus of FIG. 21, and a second connector connected to a body tissue conduit in the patient, in a still later stage of a procedure in accordance with the present invention;

FIG. 25 is a simplified sectional view of the connector of FIG. 17 in yet another configuration, illustrated with the graft tissue conduit of FIG. 5, the apparatus of FIG. 21, and a second connector connected to a body tissue conduit in the patient, in a still later stage of a procedure in accordance with the present invention;

FIG. 26 is a view similar to FIG. 23 of the connector of FIG. 2 in the configuration of FIG. 8, illustrated with the graft tissue conduit of FIG. 5 and the second connector connected to the body tissue conduit of FIG. 23, in a yet still later stage of a procedure in accordance with the present invention;

FIG. 27 is a view similar to FIG. 24 of the connector of FIG. 10 in the configuration of FIG. 15, illustrated with the graft tissue conduit of FIG. 5 and the second connector connected to the body tissue conduit of FIG. 24, in a yet still later stage of a procedure in accordance with the present invention;

FIG. 28 is a view similar to FIG. 25 of the connector of FIG. 17 in the configuration of FIG. 19, illustrated with the graft tissue conduit of FIG. 5 and the second connector connected to the body tissue conduit of FIG. 25, in a yet still later stage of a procedure in accordance with the present invention;

FIG. 29 is a perspective view of another illustrative embodiment of apparatus for use in delivering and deploying a first connector in accordance with the present invention;

FIG. 30 is a top elevational view of the apparatus of FIG. 29;

FIGS. 31 and 32 are planar developments of the structure of still other illustrative embodiments of a first connector constructed in accordance with the present invention; and

FIG. 33 is a planar development of the structure of an illustrative embodiment of a second connector in accordance with the present invention.

### DETAILED DESCRIPTION OF THE INVENTION

Although the invention has other possible uses, the invention will be fully understood from the following explanation of its use in providing a bypass around an obstruction in a patient's vascular system.

In some embodiments of the present invention, an incision having a controlled, predetermined length may be made in one or both of a target vessel and a graft conduit prior to creating an anastomosis between the target vessel and the graft conduit. (It should be noted that, although apparatus and methods for making a controlled-length incision will be described herein in relation to vessels between which an anastomosis is to be created, the controlled-length incision may be made in any tubular body fluid conduit in a patient.) Apparatus and methods for making a controlled length incision are also described, for example, in concurrently-filed, commonly-assigned U.S. patent application Ser. No. ________ (Atty. Docket No. 293/052), filed Oct. 3, 2003, which is hereby incorporated by reference herein in its entirety.

The vessel incision tool of the present invention may be used in a manner similar to that of a suture needle. The vessel incision tool includes a sharp tip for insertion into the side wall of a vessel. By inserting the tip into the side wall of the vessel, a first small hole is created in the side wall. The tool may include a small recess that provides a physician with tactile and visual feedback when the tip has been inserted far enough into the vessel. The vessel incision tool may then be rotated upwards until the tip emerges from within the vessel through the side wall. This rotation of the tool creates a second small hole in the side wall of the vessel. The distance between the first and second holes may be based on the length of the tip. For example, for a tool having a straight tip that is about 2.75 mm to about 3.00 mm in length, the resulting distance between the two small holes will be from about 2.75 mm to about 3.25 mm.

The vessel incision tool includes a sharp cutting blade to cut from hole to hole in the vessel. The tool may be rotated upward using a suture needle-like motion, thereby driving the cutting blade from the first small hole to the second small hole in the side wall. The tool is then removed from the vessel, and the incision is complete. The length of the incision may be altered to fit, for example, a connector that will be deployed at the site of the incision. For example, the perimeter of the incision created using the apparatus and methods described hereinabove may be identical to the perimeter of the expanded connector installed at the incision.

The controlled length incision created using the vessel incision tool of the present invention is effective for anastomoses such as, for example, anastomoses involving non-round connectors and for sutured anastomoses. Creating the incision allows a physician to inspect the incision and surrounding vessel for quality and disease prior to, for example, installing a connector at the site of the incision or sewing a graft to the vessel.

The tip and blade of the vessel incision tool may be constructed of any suitable rigid material, such as, for example, stainless steel. The blade of the vessel incision tool may be of any suitable shape, such as, for example, straight, curved, or any other shape suitable for cutting from the first hole to the second hole in the vessel side wall. The blade of the vessel incision tool may be at any suitable angle with respect to both the tip and the handle of the tool for different target sites.

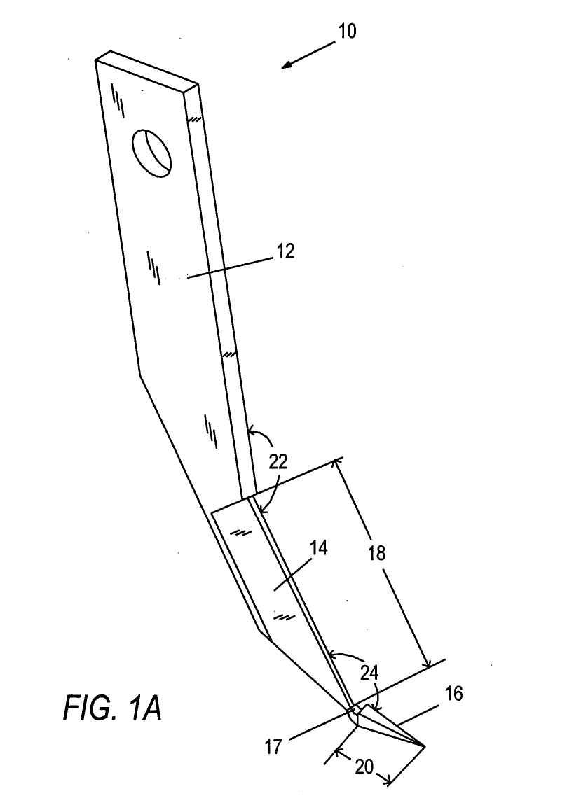

FIG. 1A is a perspective view of an illustrative vessel incision tool 10 in accordance with the present invention. Vessel incision tool 10 may include a handle 12, blade 14, and tip 16. Tool 10 may include a recess 17 between tip 16 and blade 14 that indicates to a physician when tip 16 has been inserted far enough into a vessel. Blade 14 and tip 16 have respective lengths 18 and 20. In one illustrative example, length 18 may be approximately 6.4 mm, and length 20 may be approximately 2.5 mm. Handle 12 and blade 14 form an angle 22, and blade 14 and tip 16 form an angle 24. As described hereinabove, angles 22 and 24 may be adjusted to accommodate different target sites.

FIGS. 1B-1E illustrate a method for creating a controlled length incision in a vessel using, for example, vessel incision tool 10 of FIG. 1A. As shown in FIG. 1B, sharp tip 16 of tool 10 may be inserted through side wall 32 of vessel 30. Tip 16 may be inserted through side wall 32 as far as recess 17. The insertion of tip 16 through side wall 32 results in the creation of a first hole 34. To achieve the orientation of FIG. 1C, tool 10 may be further rotated such that sharp tip 16 is forced through side wall 32, thereby creating a second hole 36. The distance from second hole 36 to first hole 34 is approximately equal to length 20 (FIG. 1A) of tip 16. After tip 16 has emerged from within vessel 30, tool 10 may be rotated upward such that cutting blade 14 cuts from first hole 34 to second hole 36, as shown in FIG. 1D. This results in the creation of an incision 38 having a controlled, predetermined length 40, as shown in a top down view in FIG. 1E. As stated hereinabove, length 40 of incision 38 may be approximately equal to length 20 (FIG. 1A) of tip 16. (It should be noted that the incision created in the vessel, and any other incision referred to herein, may also be referred to as an “aperture.”)

FIGS. 1F and 1G are perspective views of another illustrative vessel incision tool 50 in accordance with the present invention. FIG. 1F shows tool 50 as rotated 90-degrees into the page from the orientation of FIG. 1G. Vessel incision tool 50 has a certain curvature which may be constant or may vary along the length of the tool. The curvature of tool 50 may be such that tool 50 is suitable for a particular target site. Vessel incision tool 50 may include a handle 52, blade 54, and tip 56. Tool 50 may include a recess 57 between tip 56 and blade 54 that indicates to a physician when tip 56 has been inserted far enough into a vessel. Blade 54 and tip 56 of vessel incision tool 50 may be constructed of any suitable material such as, for example, stainless steel.

Vessel incision tool 50 may be used to create a controlled length incision in a vessel in a manner substantially similar to that described hereinabove in connection with FIGS. 1B-1E.

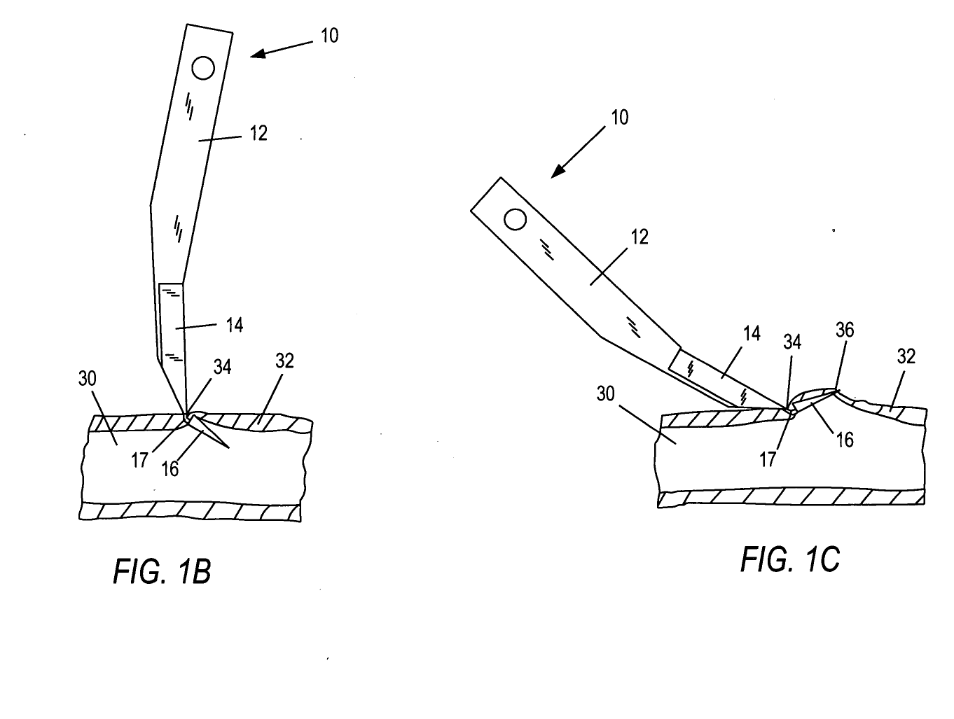

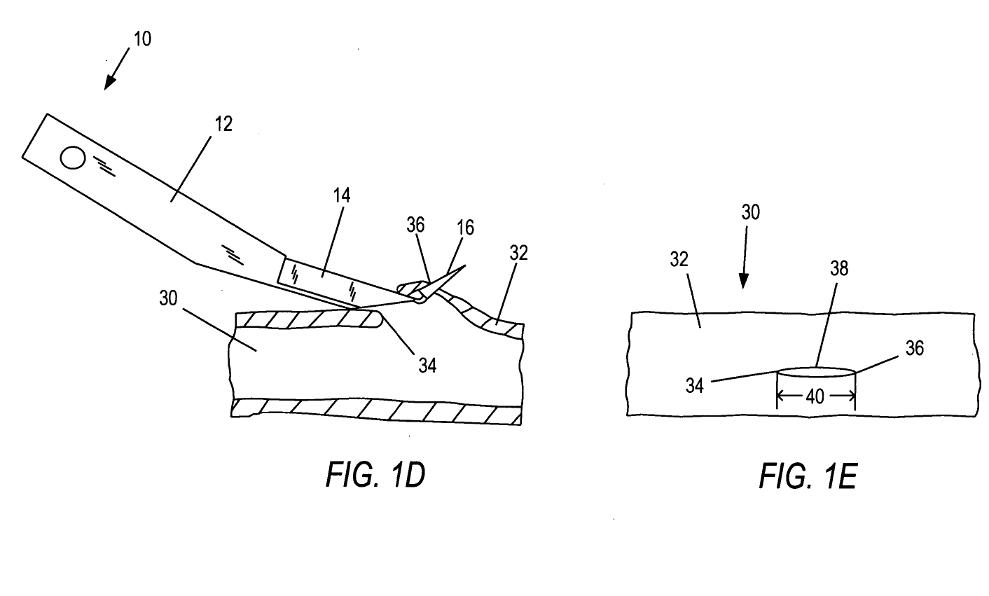

FIG. 2 shows a planar development of what is actually, preferably, an integral, one-piece (unitary), annular, self-expanding cellular exterior connector 100. In particular, the left and right edges of the structure shown in FIG. 2 are actually, preferably, joined to and integral with one another. Thus, the actual structure of connector 100 is as shown in FIG. 3, although FIG. 2 is useful to more clearly reveal certain details of various features of connector 100.

A particularly preferred material for self-expanding connector 100 is nitinol. Other examples of suitable materials include tantalum, tungsten, and platinum. Connector 100 may be advantageously produced by starting with a single, unitary tube, such as a hypotube, and removing selected material until only the structure shown in FIG. 3 remains. For example, laser cutting may be used to remove material from the starting tube in order to produce connector 100.

Connector 100 may be described as including annularly spaced cell portions 112. According to one embodiment, connector 100 includes six repeating cell portions 112. The connector may have fewer or more than six of cell portions 112, depending on the axial length and perimeter of the tube used to manufacture connector 100 and the resulting perimeter desired. Alternatively, the structure of connector 100 may have different configurations of cells and geometries.

Each cell portion 112 includes a pair of annularly spaced members 120. The distal ends of members 120 are connected to one another at an annularly extending member 122. A pair of members 120 and a member 122 define the distal portion 114 of each cell portion 112. Each cell portion 112 also includes a pair of annularly spaced members 128. The proximal ends of members 128 are connected to one another at an annularly extending member 130, which is preferably curved proximally. A pair of members 128 and a member 130 define the proximal portion 118 of each cell portion 112. In each cell portion 112, the most distal point of member 122 and the most proximal point of member 130 may typically be separated by a distance 113 with a length in a range from about 0.106 inches to about 0.111 inches. (It should be noted that the length of distance 113 includes the width of member 122 and the width of member 130.) Each cell portion 112 also includes a pair of annularly spaced members 136. The proximal ends of members 136 are connected to the distal ends of members 128, and the distal ends of members 136 are connected to the proximal ends of members 120. A pair of members 136 defines the medial portion 116 of each cell portion 112. Medial portions 116 of annularly adjacent cell portions 112 may typically be separated by a distance 117 with a length in a range from about 0.046 inches to about 0.056 inches.

A cell portion 112 may be connected to an annularly adjacent cell portion 112 by members 138 and 140. Connecting annularly adjacent cell portions 112 at more than one location (e.g., using two members 138 and 140) improves the rigidity of connector 100. For example, with only one connection between annularly adjacent cell portions 112, a cell portion 112 has a tendency to twist with respect to the two annularly adjacent cell portions. However, with two connections between annularly adjacent cell portions 112 (e.g., members 138 and 140), a cell portion 112 is prevented from twisting with respect to the two annularly adjacent cell portions. Although two connections are shown in FIG. 2, it will be appreciated that annularly adjacent cell portions 112 may be connected to one another using any number of connections.

Some or all of the distal portions 114 of the cell portions 112 may include a tissue holding feature that in this case includes a distal member 124 that has a barb-like free end portion 126 that is sharply pointed and that points toward proximal portion 118. Distal member 124 may be connected to annularly extending member 122. A typical distal member 124 may have a length 125 in a range from about 0.047 inches to about 0.057 inches. (It should be noted that length 125 includes the width of member 122.) However, the dimensions of distal member 124 may be altered according to the wall thickness of the conduits to be joined. Each of distal members 124 is deflectable radially outward from the remainder of the structure of connector 100, as shown, for example, in FIG. 3.

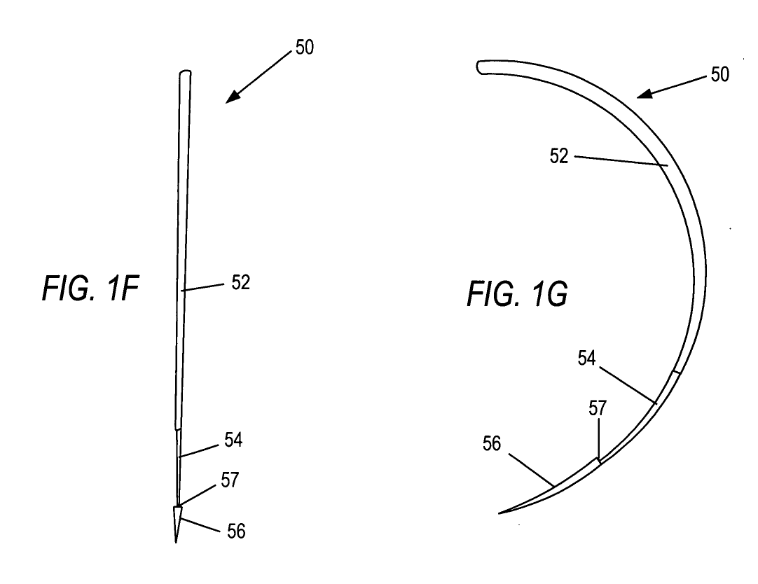

The above-mentioned outward deflection of distal members 124 may be produced by putting connector 100 on a mandrel and prying members 124 radially outward. Following deflection of members 124, an initial distal perimeter 111 and an initial proximal perimeter 119 may be defined by the tips of distal members 124 and proximal members 130, respectively. These perimeters are “initial” perimeters in contrast to the “expanded” perimeters defined by the tips of members 124 and 130 following expansion of connector 100 (see, for example, FIG. 4). The initial distal perimeter 111, for example, may be defined by the pointed tips of free end portions 126. The perimeters defined by distal members 124 and proximal members 130 of connector 100 are one aspect of the invention that allows the members to engage and/or penetrate the two conduits to be connected upon expansion of the connector.

Connector 100 also typically requires other processing appropriate for an implantable device such as, for example, polishing, passivation, cleaning, and sterilizing.

FIG. 4 illustrates the expanded condition of connector 100. Connector 100 is formed in such a way that it is annularly self-expandable. It is to be understood that the deflection of members 124 described above may instead be a result of the expansion of connector 100.

The annular enlargeability of connector 100 is provided by annularly self-expanding cell portions, such as cell portions 112 described above. In this way, connector 100 is annularly enlargeable by the annular expansion of some or all of cell portions 112.

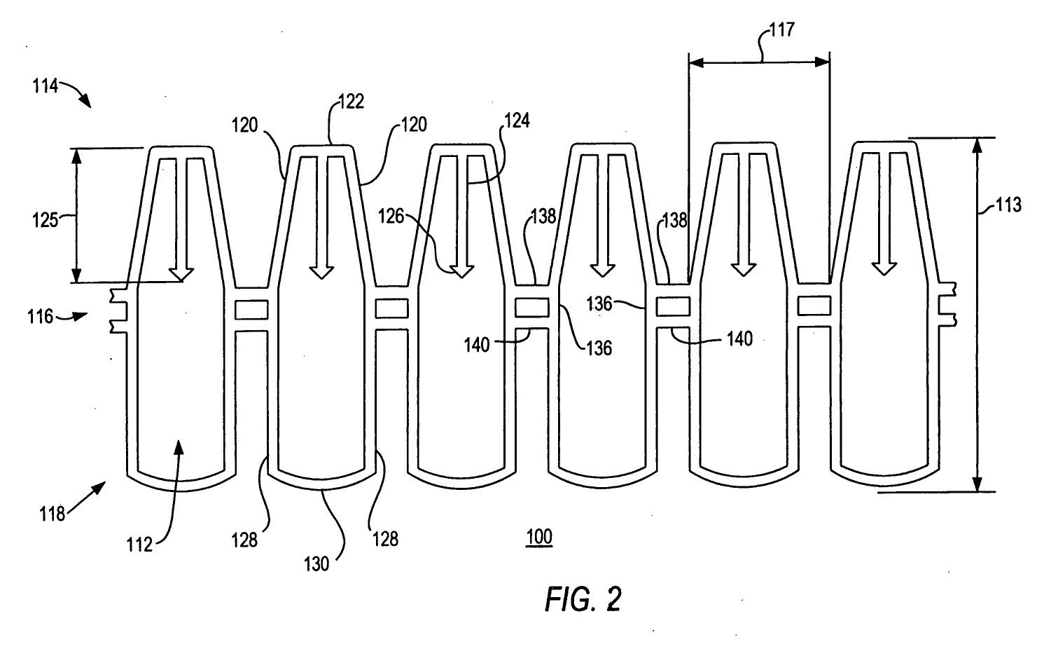

As connector 100 is enlarged, it is desirable for the distal portion 114 and the proximal portion 118 to expand radially outward to form greater perimeters than the perimeter associated with medial portion 116. Following expansion of distal portion 114, an “expanded” distal perimeter 115 may be defined by the tips of distal members 124. This perimeter is an “expanded” perimeter in contrast to the “initial” distal perimeter 111 as shown in FIG. 4, for example. Expanded distal perimeter 115, for example, may be defined by the pointed tips of free end portions 126. Furthermore, following expansion of proximal portion 118, an “expanded” proximal perimeter 131 may be defined by the proximal tips of members 130. This perimeter is an “expanded” perimeter in contrast to the “initial” proximal perimeter 119 as shown in FIG. 3, for example. The overall annular expansion of connector 100 along with the relatively greater enlargement of distal portion 114 and proximal portion 118 together decrease the axial spacing distance between members 130 and members 122 (and, therefore members 124), resulting in the axial spacing 137 shown in FIG. 4. Spacing 137 may vary depending on the wall thicknesses of the two conduits to be joined by connector 100. Unlike other anastomosis schemes, cellular exterior connector 100 substantially resides on the outside of a first graft conduit along its axis. Attachment of connector 100 to a first conduit will now be described with reference to FIGS. 5-9.

A first conduit 200 is subsequently mounted to connector 100 about an aperture 202 made in the side wall of the first conduit 200. As shown in FIG. 5, for example, interior surface 204 of first conduit 200 defines a lumen 208 for providing fluid flow between first end portion 209, second end portion 210, and aperture 202 of first conduit 200. First conduit 200 may be natural body tissue (e.g., a length of the patient's saphenous vein harvested for use as a graft, a partly severed internal mammary artery, etc.), an artificial graft (e.g., as shown in Goldsteen et al. U.S. Pat. No. 5,976,178, which is hereby incorporated by reference herein in its entirety), or a combination of natural and artificial conduits (e.g., a length of natural conduit disposed substantially concentrically inside a length of artificial conduit).

Aperture 202 with a perimeter 203 may be made in first conduit 200 between interior surface 204 and exterior surface 206 at a location spaced from end portions 209 and 210, and may be made by the apparatus and methods described above relating to a controlled-length incision, or by mechanical dilation, or a combination of both. In one example, an initial aperture is made by cutting first conduit 200 with a blade and then dilating the aperture using a dilator. For example, an incision may be made in conduit 200 that is about 80% of the length of the ostium to be defined by loading it on connector 100 having an expanded perimeter 115. However, the size of the initial incision may be selected based upon the elastic characteristics of first conduit 200 or on any other factor related to the size and perimeter of the aperture in the first conduit. Other examples of methods and apparatus for creating an aperture in a side wall of a conduit are described, for example, in published Patent Cooperation Treaty (“PCT”) patent application publication No. WO 01/39672, published Jun. 7, 2001, which is hereby incorporated by reference herein in its entirety. Such methods and apparatus may also be used to create an aperture in a second conduit (e.g., a patient's body tissue conduit).

The size of perimeter 203 of aperture 202 in first conduit 200 is an important consideration. (It is understood that the description concerning aperture 202 may also be applicable to the second aperture made in the second conduit.) If the aperture is too large, then a satisfactory hemodynamic seal may not be created between the two conduits. Conversely, if the aperture is too small, one or more of the following undesirable effects may occur: the conduit walls may tear excessively when connector 100 is expanded, or the conduit may constrict expansion of the connector. (When making the opening in the second conduit, the opening may not permit a delivery tool and/or deformed first connector to be inserted therethrough if it is too small.) Which of these above effects occurs is determined in part by tissue quality, the dimensions of the apparatus being used, and the expansion pressure of the connector.

The perimeter of the aperture in the conduit should preferably be sized such that attachment to and/or expansion of connector 100 does not cause significant additional tearing of the wall to expand the periphery of the opening. Rather, it is generally desirable that the expansion of the aperture to accommodate the expanded connector is achieved within the elastic expansion range of the conduit walls. The elastic expansion is important because distal members 124 of connector 100 engage the walls of first conduit 200 as the connector structure deforms and expands. If the conduit tears a significant amount, (e.g., at the locations of engagement with distal members 124 rather than elastically expanding), it is possible that the desired tension created in the conduit between distal members 124 would be relieved, which may prevent the creation of a seal between the conduits being joined. As an example, the perimeter of the aperture in the conduit should be between about 70-80% of the size of the expanded distal perimeter of the connector. This will preferably allow the elasticity of the conduit tissue to assist in creating a seal between the conduits as they are stretched to the perimeter of the expanded connector.

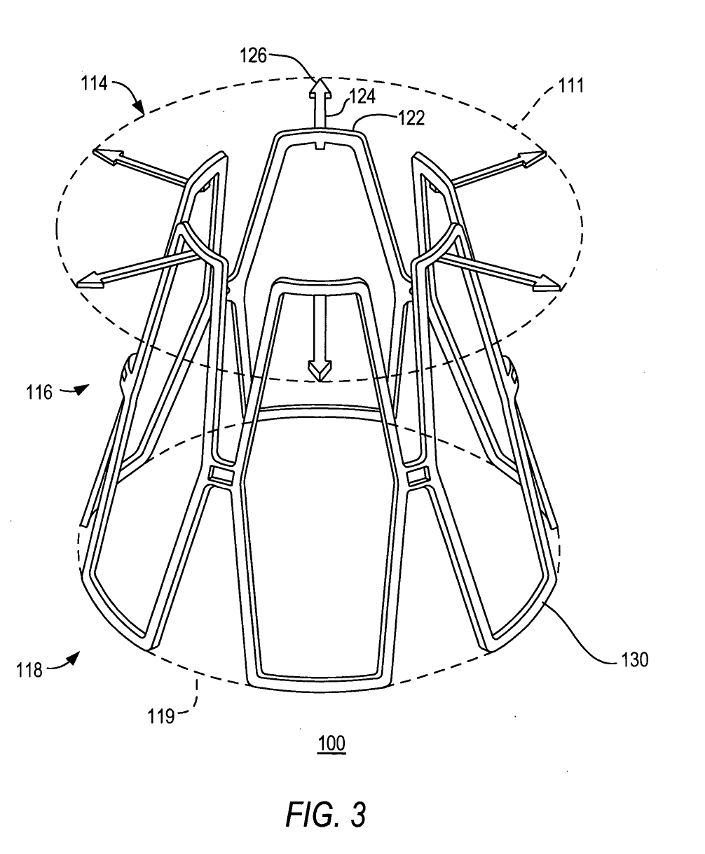

For attachment, first conduit 200 may be positioned below proximal portion 118 of expanded connector 100 such that aperture 202 made in the side wall of first conduit 200 faces and is substantially parallel to expanded distal perimeter 115. As illustrated in FIG. 6, periphery 203 of aperture 202 is advanced in the direction of arrow 212 through the hollow of connector 100 beyond distal portion 114, and then in the direction of arrows 214 beyond annularly extending members 122 and distal members 124 of connector 100.

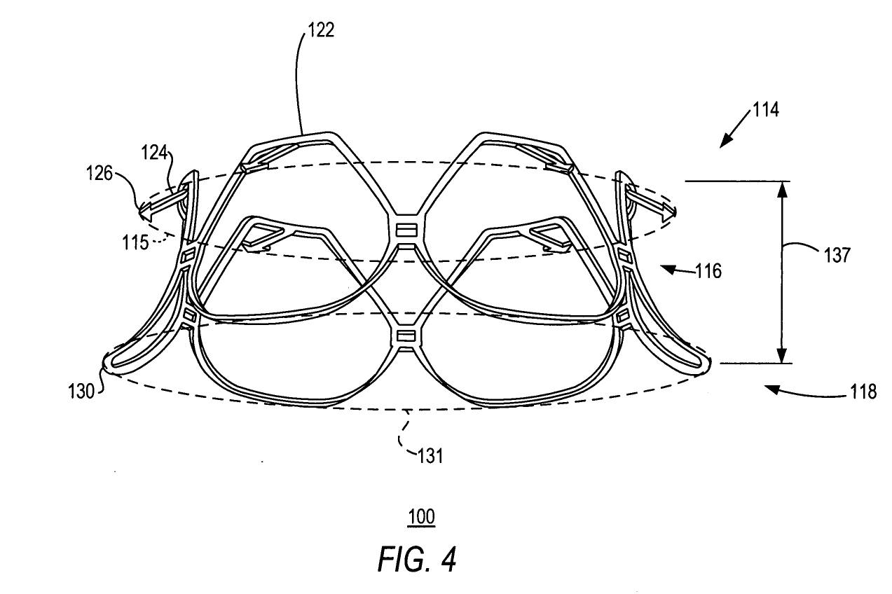

FIG. 7 illustrates that first conduit 200 is advanced until exterior surface 206 around periphery 203 of aperture 202 envelops expanded distal perimeter 115 defined by the pointed tips of free end portions 126 of connector 100. Once periphery 203 of aperture 202 has passed beyond distal members 124 of connector 100, it rests on the tips of free end portions 126. This is because expanded distal perimeter 115 is larger than aperture 202, thereby holding the aperture in place at distal portion 114. Preferably, the clearance between perimeter 203 and expanded distal perimeter 115 is minimal to ensure that distal members 124 engage first conduit 200 evenly around periphery 203 of aperture 202. Subsequently, first conduit 200 is retained in position (e.g., with an atraumatic grasping instrument).

With continued reference to FIG. 7, periphery 203 of aperture 202 in first conduit 200 is placed about connector 100. More particularly, conduit 200 is positioned so that distal members 124 penetrate and pass through the side wall of conduit 200 from exterior surface 206 to interior surface 204 as a result of, for example, compressing the graft against the tips of free end portions 126 with a tool 300 (e.g., the vein piercing tool described in John Logan et al. U.S. patent application Ser. No. 09/587,112, filed Jun. 2, 2000, which is hereby incorporated by reference herein in its entirety), thereby forcing the free end portions to pierce through the graft wall. The sharpened tips of free end portions 126 of distal members 124 facilitate penetration of conduit 200 by members 124. The blunt rear surfaces of free end portions 126 resist withdrawal of members 124 from conduit 200 after members 124 have penetrated the conduit, like a barb. Conduit 200 may be additionally or alternatively directly sutured to connector 100. Alternatively, first conduit 200 may be secured to connector 100 by, for example, pinching, inverting, clinching, stretching, or any other suitable manner of attaching the graft to the connector, with or without glues, clips, or any other connector elements.

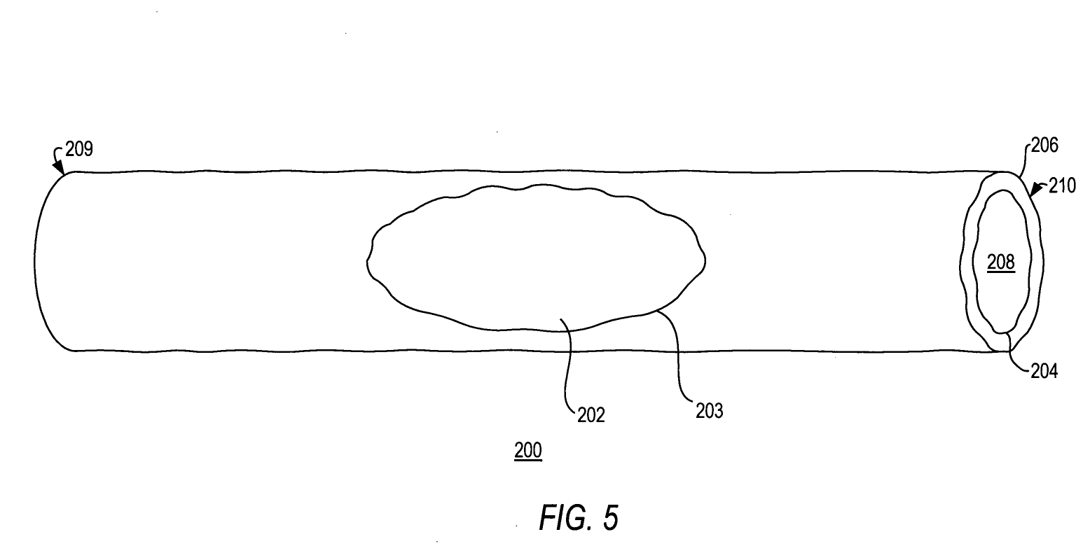

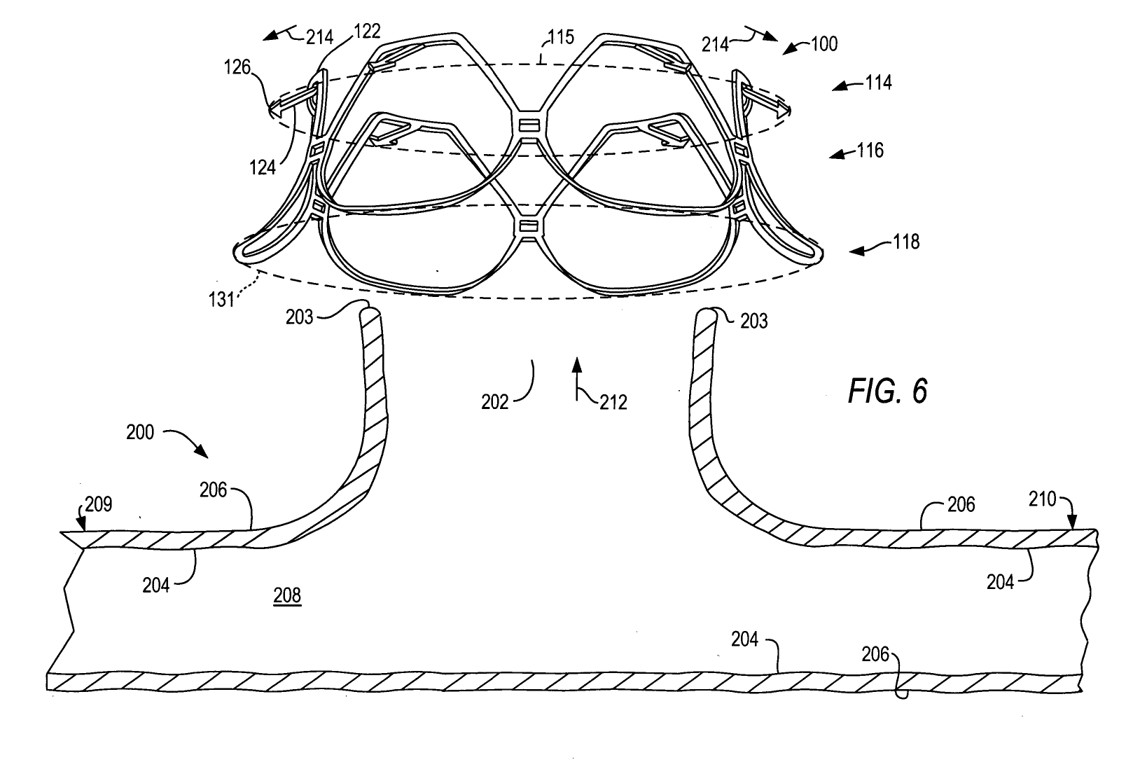

As an alternative to securing first conduit 200 to connector 100 in its expanded condition, connector 100 may expand to its expanded condition after first conduit 200 has been secured to the connector structure in its non-expanded form (see, e.g., FIG. 3 with deflected members 124). Either way, once the conduit is secured to the connector, a portion of each distal member 124 that includes free end portion 126 may be bent back such that free end portion 126 points substantial parallel to members 136 of medial portion 116 in order to reduce expanded distal perimeter 115, as shown in FIG. 8, for example. Following the expansion and bending of members 124 and annular expansion of members 130, axial spacing 137 may be defined therebetween as shown in FIGS. 8 and 9.

It will be appreciated that the perimeter of the ostium created by connector 100 through aperture 202 is not defined by perimeter 203, but instead by a smaller ostium perimeter 201, because perimeter 203 has been passed beyond distal members 124. Ostium perimeter 201 may generally be understood as having a substantially axial length 250 and a substantially trans-axial length 252. Perimeter 203 of first conduit 200 may be engaged with connector 100 at the points where members 124 meet members 122. Spacing 137 may vary depending on the wall thicknesses of the two conduits to be joined by connector 100. This aspect of the invention will be described in greater detail herein.

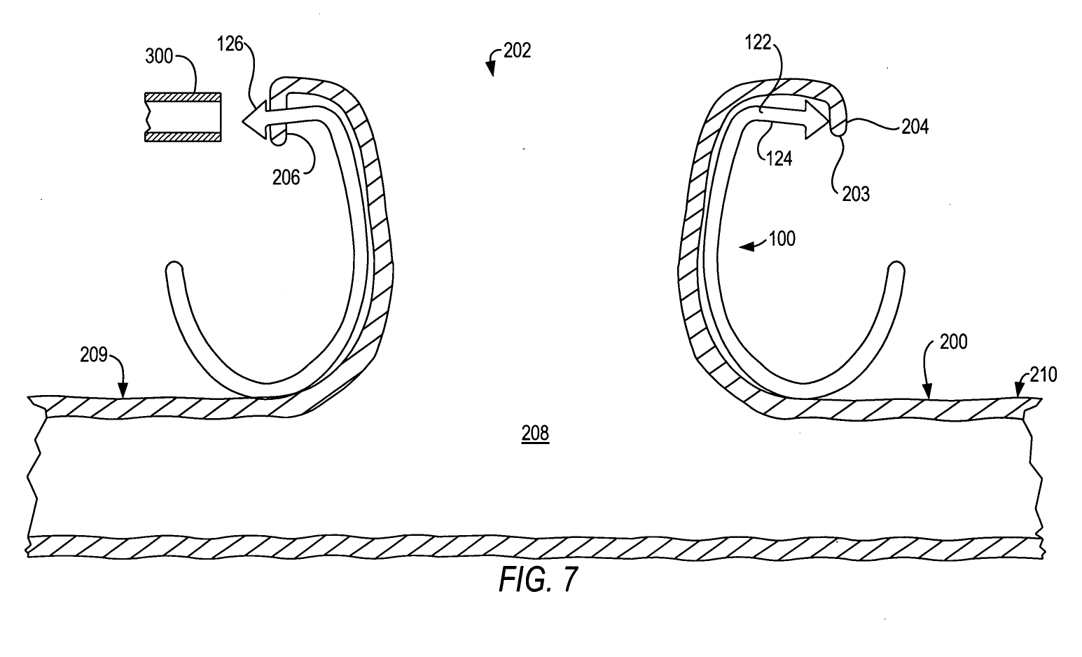

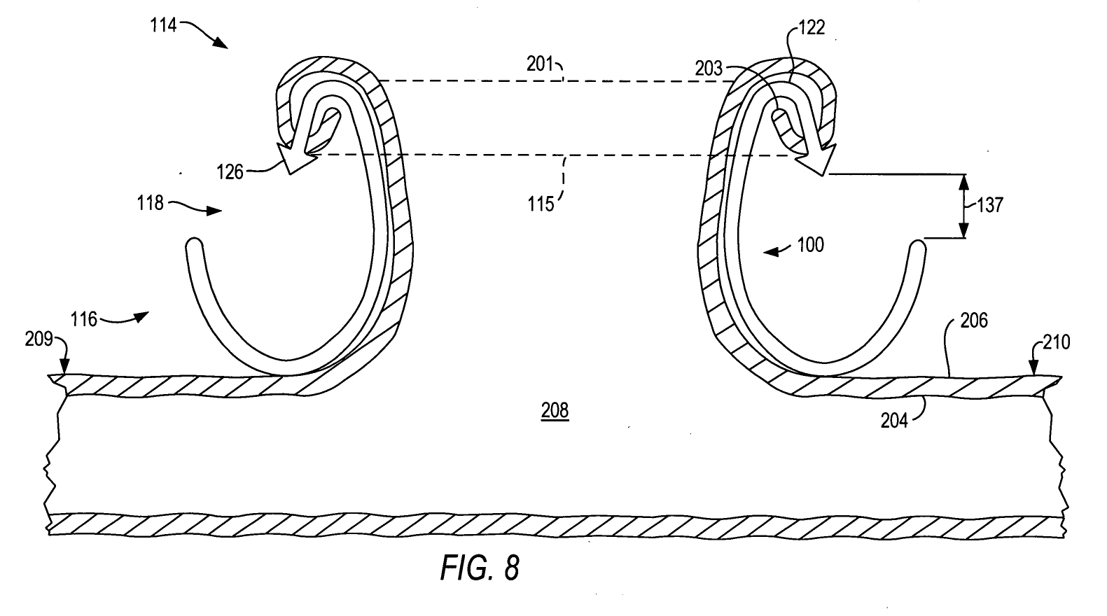

In another preferred embodiment of the self-expanding exterior connector of the present invention, FIG. 10 shows a planar development of what is actually, preferably, an integral, one-piece (unitary), annular, self-expanding tubular exterior connector 1100. In particular, the left and right edges of the structure shown in FIG. 10 are actually, preferably, joined to and integral with one another. Thus, the actual structure of connector 1100 is as shown in FIG. 11, although FIG. 10 is useful to more clearly reveal certain details of various features of connector 1100.

A particularly preferred material for self-expanding connector 1100 is nitinol. Connector 1100 may be advantageously produced by starting with a single, unitary tube, such as a hypotube, and removing selected material until only the structure shown in FIG. 11 remains. For example, laser cutting may be used to remove material from the starting tube in order to produce connector 1100.

Self-expanding tubular exterior connector 1100 may be described as including axially spaced expandable finger portion pairs 1110. According to one embodiment, connector 1100 includes four axially spaced finger portion pairs 1110. Connector 1100 may also include expandable end finger portion pairs 1020 axially spaced from particular finger portion pairs 1110. According to the embodiment shown in FIGS. 10-16, connector 1100 includes two end finger portion pairs 1120, one at each end of the tubular structure of the connector. The connector may have fewer or more than four finger portion pairs 1110 and may have two, none, or just one end finger portion pair 1120, depending on the axial length and perimeter of the tube used to manufacture connector 1100 and the resulting perimeter desired. Alternatively, the structure of connector 1100 may have different configurations of fingers and geometries.

Each axially spaced finger portion pair 1110 includes annularly spaced fingers 1112a and 1112b. The distal ends of fingers 1112a and 1112b of each finger portion pair 1110 are annularly spaced from each other by an initial trans-axial opening distance 1117. The distal ends of fingers 1112 and a trans-axial opening distance define the distal portion 1114 of each finger portion pair 1110. Each end finger portion pair 1120 includes end fingers 1122a and 1122b. The distal ends of end fingers 1122a and 1122b are connected to one another at an end member 1123. The distal ends of fingers 1122 and an end member 1123 define the distal portion 1114 of each finger portion pair 1120. The proximal ends of fingers 1112 of each of the finger portion pairs 1110 and the proximal ends of fingers 1122 of each of the finger portion pairs 1120 are connected to a common proximal body 1130 at junctions 1132. The proximal ends of fingers 1112, the proximal ends of fingers 1122, junctions 1132, and proximal body 1130 define the proximal portion 1118 of connector 1100. Any adjacent finger pair portions 1110 or 1120 are axially spaced from each other by an initial axial finger spacing distance 1119, and finger pair portions 1120 are axially spaced from each other by an initial axial opening distance 1121. Each one of fingers 1112 and 1122 may typically have a width 1140 in a range from about 0.30 inches to about 0.40 inches. The single tube from which connector 1100 is produced may typically have an initial perimeter 1102 with a length in a range from about 0.6030 inches to about 0.7030 inches and an initial axial length 1104 in a range from about 0.1750 inches to about 0.2750 inches.

The distal ends of some or all of fingers 1112 may include a tissue holding feature that, in this case, includes a distal member 1124 that has a free end portion 1126 that is sharply pointed and that points away from proximal portion 1118. One or both of end members 1123 may also include a similar tissue holding feature, although end members 1123 may each have a different type of tissue holding feature than the other end member and fingers 1112, if desired. A typical distal member 1124 may have a length 1125 in a range from about 0.05 inches to about 0.06 inches. However, the dimensions of distal member 1124 may be altered according to the wall thicknesses of the conduits to be joined. Each of distal members 1124 is deflectable outward from the remainder of the structure of connector 1100, as shown, for example, in FIG. 11.

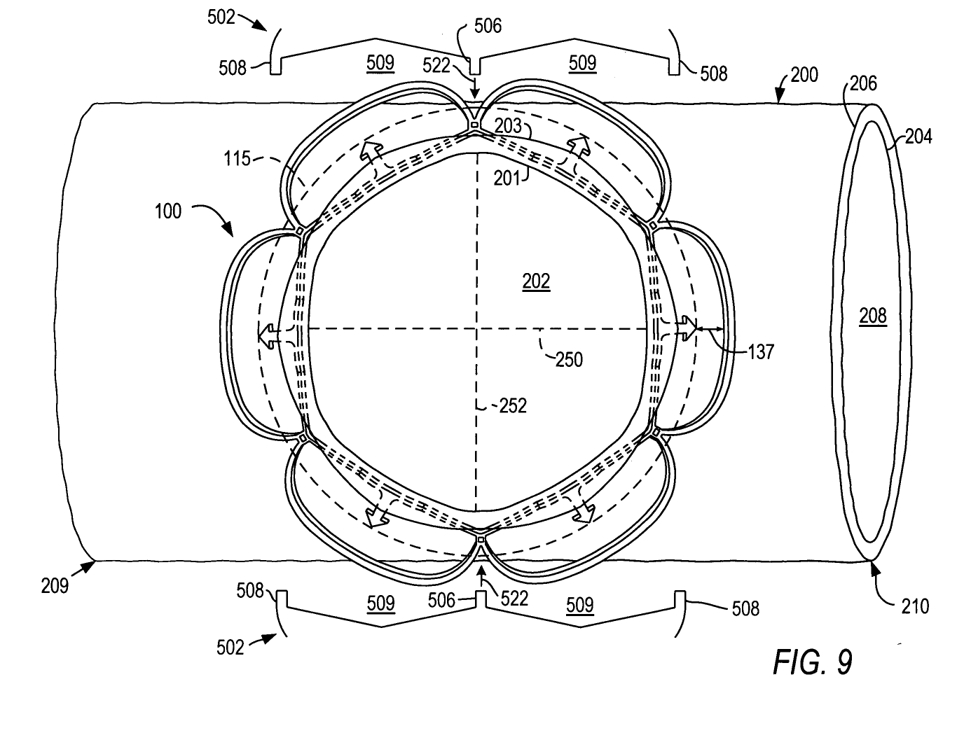

The above-mentioned outward deflection of distal members 1124 may be produced by putting connector 1100 on a mandrel and prying members 1124 outward, as described above with respect to members 124. Following deflection of members 1124, an initial distal perimeter 1111 may be defined by the tips of distal members 1124. This perimeter is an “initial” perimeter in contrast to the “expanded” distal perimeter defined by the tips of members 1124 following expansion of connector 1100 (see, for example, FIG. 12). The initial distal perimeter 1111, for example, may be defined by the pointed tips of free end portions 1126. The perimeters defined by distal members 1124 of connector 1100 are one aspect of the invention that allows the members to engage and/or penetrate the two conduits to be connected upon expansion of the connector.

Connector 1100 also typically requires other processing appropriate for an implantable device such as, for example, polishing, passivation, cleaning, and sterilizing.

FIG. 12 illustrates the expanded condition of connector 1100. Connector 1100 is formed in such a way that it is annularly and axially self-expandable. It is to be understood that the deflection of members 1124 described above may instead be a result of the expansion of connector 1100. It is also to be understood that in another embodiment, connector 1100 may be substantially annularly continuous but with the left and right edges of the structure shown in FIG. 10 separate from one another, although substantially close, while at least one end member 1123 allows the connector to remain a one-piece (unitary) structure.

The annular enlargeability of connector 1100 is provided by annularly self-expanding finger portion pairs 1110 which provide expanded trans-axial opening distances 1127 that are greater than initial trans-axial opening distances 1117. The axial enlargeability of connector 1100 is provided both by self-expanding finger portion pairs 1110, which provide expanded axial finger spacing distances 1129 that are greater than initial axial finger spacing distances 1119, and by axially self-expanding finger portion pairs 1120 which provide an expanded axial opening distance 1131 that is greater than initial axial opening distance 1121. In this way, connector 1100 is annularly and axially enlargeable by enlarging some or all of the distances between finger portion pairs 1110 and/or finger portion pairs 1120.

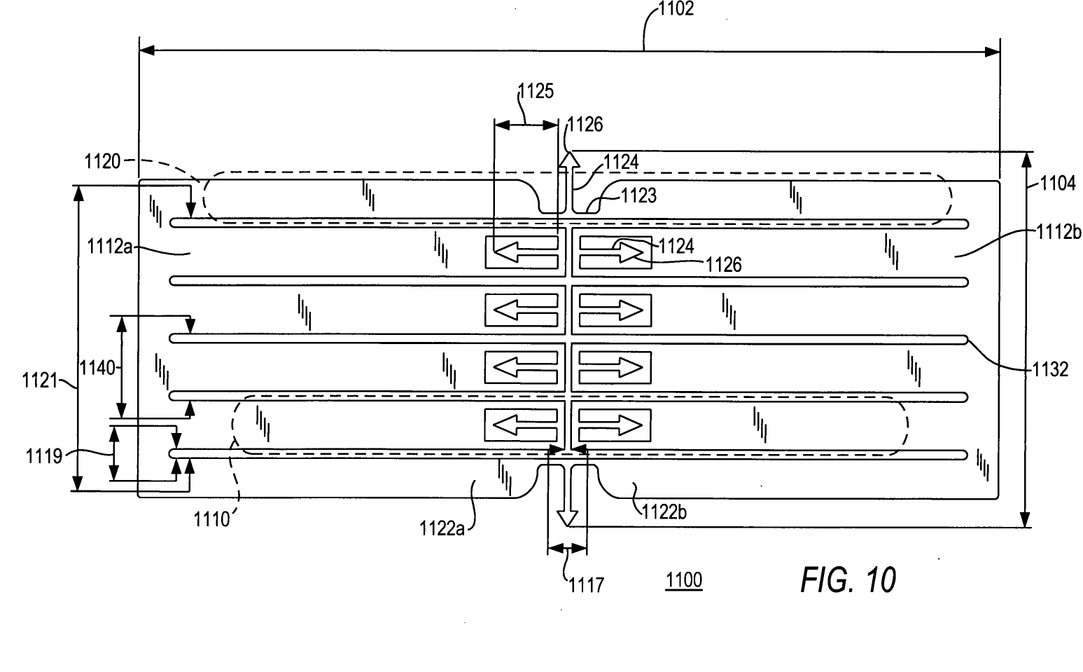

Following expansion of finger portion pairs 1110 and 1120, an “expanded” distal perimeter 1115 is defined by the tips of distal members 1124. This perimeter is an “expanded” perimeter in contrast to the “initial” distal perimeter 1111 as shown in FIG. 11. Expanded distal perimeter 1115, for example, may be defined by the pointed tips of free end portions 1126. In the embodiment shown in FIG. 12, for example, expanded trans-axial opening distances 1127 may be greater between fingers 1112 which are further away from end finger portions 1120 such that expanded distal perimeter 1115 is trans-axially wider at certain points than others in order to be used with various sizes and shapes of apertures in the conduits to be connected. The overall expansion of connector 1100 may decrease the spacing distance between fingers 1112 and their end portions 1126, resulting in the spacing 1137 shown in FIG. 12. Spacing 1137 may vary depending on the wall thicknesses of the two conduits to be joined by connector 1100. Attachment of connector 1100 to a first conduit will now be described with reference to FIGS. 13-16.

For attachment, first conduit 200 may be positioned within the hollow lumen of expanded tubular connector 1100, such that aperture 202 made in the side wall of first conduit 200 faces and is substantially parallel to expanded distal perimeter 1115. As illustrated in FIG. 13, periphery 203 of aperture 202 is advanced in the direction of arrow 212 through the opening of connector 1100 beyond distal portion 1114, and then in the direction of arrows 214 beyond each of the distal members 1124 of connector 1100.

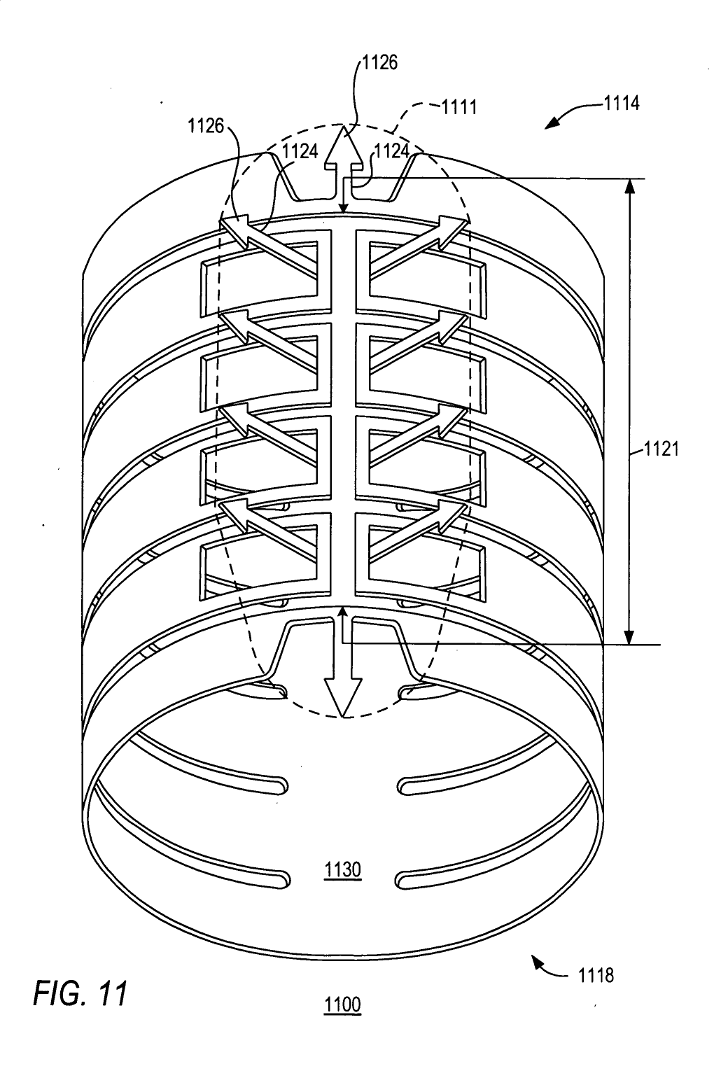

FIG. 14 illustrates that first conduit 200 is advanced until exterior surface 206 around periphery 203 of aperture 202 envelops self-expanded distal perimeter 1115 defined by the pointed tips of free end portions 1126 of connector 1100. Once periphery 203 of aperture 202 has passed beyond distal members 1124 of connector 1100, it rests on the tips of free end portions 1126. Preferably, the clearance between perimeter 203 and expanded perimeter 1115 is minimal to ensure that distal members 1124 engage first conduit 200 evenly around periphery 203 of aperture 202. Subsequently, first conduit 200 is retained in position (e.g., with an atraumatic grasping instrument).

With continued reference to FIG. 14, periphery 203 of aperture 202 in first conduit 200 is placed about connector 1100. More particularly, conduit 200 is positioned so that distal members 1124 penetrate and pass through the side wall of conduit 200 from exterior surface 206 to interior surface 204 as a result of, for example, compressing the graft against the tips of free end portions 1126 with tool 300, thereby forcing the free end portions to pierce through the graft wall. The sharpened tips of free end portions 1126 of distal members 1124 facilitate penetration of conduit 200 by members 1124. The blunt rear surfaces of enlarged free end portions 1126 resist withdrawal of members 1124 from conduit 200 after members 1124 have penetrated the conduit. Conduit 200 may be additionally or alternatively directly sutured to connector 1100. Alternatively, first conduit 200 may be secured to connector 1100 with glues, clips, or other connector elements.

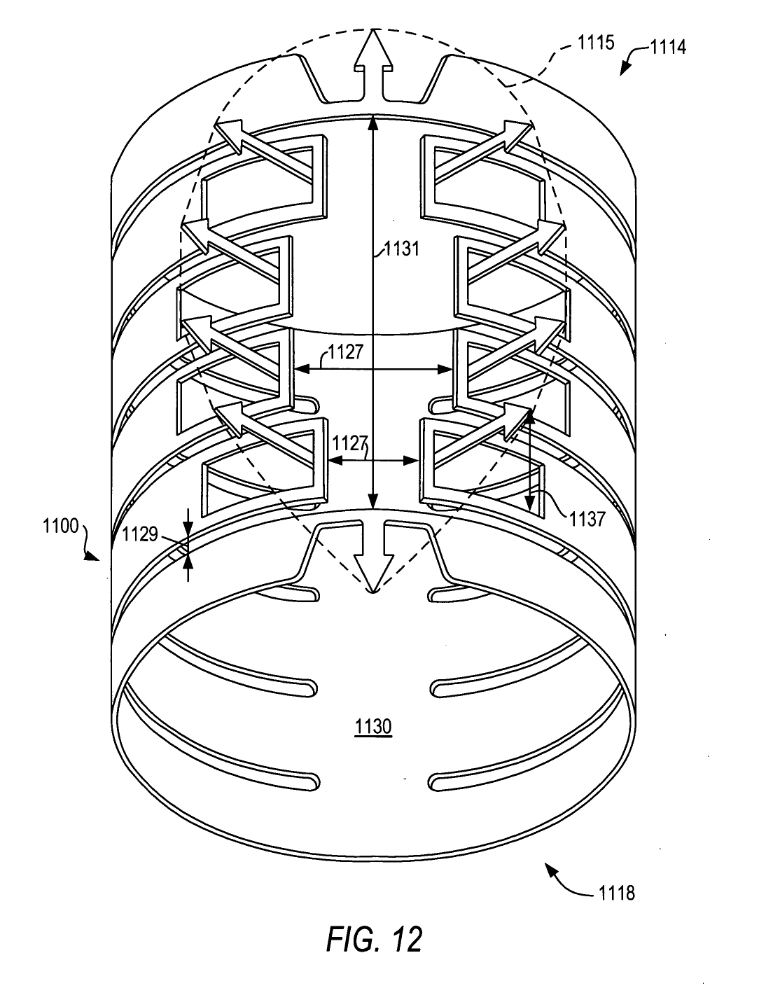

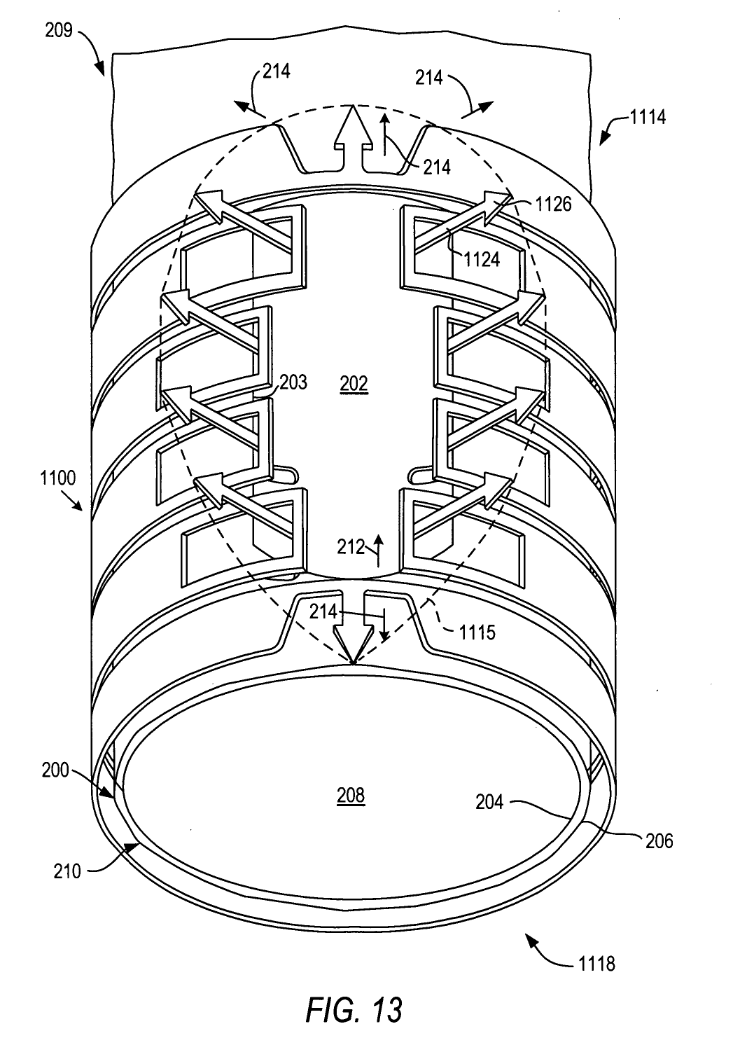

As an alternative to securing first conduit 200 to connector 1100 in its expanded condition, connector 1100 may expand to its expanded condition after first conduit 200 has been secured to the initial connector structure (see, e.g., FIG. 12 with deflected members 1124). Either way, once the conduit is secured to the connector, a portion of each distal member 1124 that includes free end portion 1126 may be bent back and down such that free end portion 1126 points substantially parallel to fingers 1112, for example, in order to reduce expanded distal perimeter 1115, as shown in FIG. 15, for example. Following expansion of portion pairs 1110 and 1120, and bending of members 1124, axial spacing 1137 may be defined therebetween as shown in FIGS. 15 and 16.

It will be appreciated that the perimeter of the ostium created by connector 1100 through aperture 202 is not defined by perimeter 203, but instead by a smaller ostium perimeter 201, because perimeter 203 has been passed beyond distal members 1124. Ostium perimeter 201 may generally be understood as having a substantially axial length 250 and a substantially trans-axial length 252. Perimeter 203 of first conduit 200 may be engaged with connector 1100 at the points where members 1124 meet members 1122. Spacing 1137 may vary depending on the wall thicknesses of the two conduits to be joined by connector 1100. This aspect of the invention will be described in greater detail herein. It is to be understood the size of perimeter 203 needed to secure first conduit 200 to connector 1100 is less than the size of perimeter 203 needed to secure first conduit 200 to connector 100, because the tissue of conduit 200 must extend up through the hollow of connector 100 (FIG. 8) as opposed to only up and over members 1124 of connector 1100 (FIG. 15).

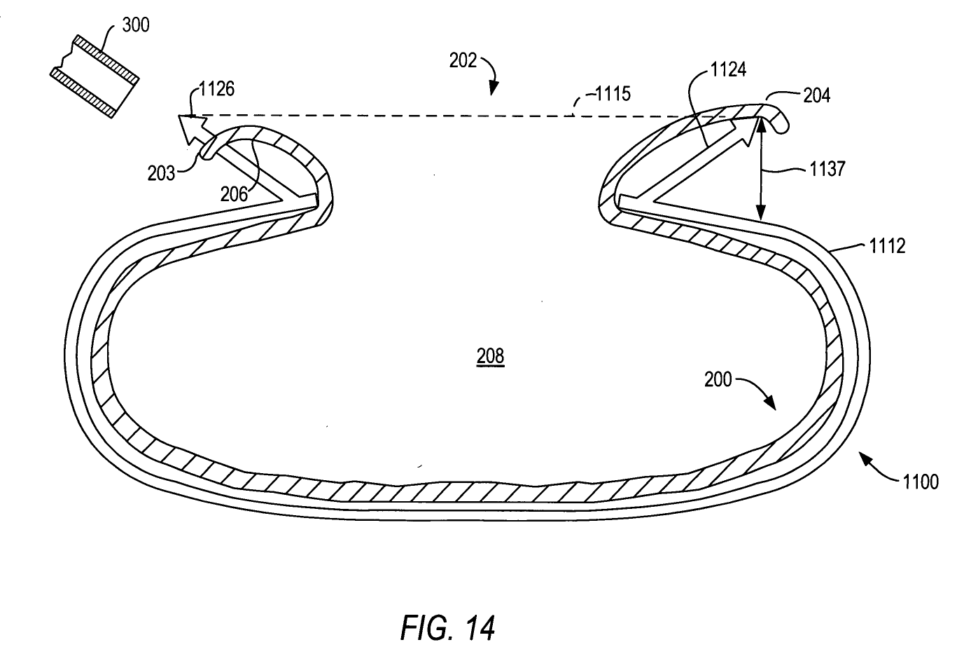

Various alternative embodiments of a self-expanding exterior connector in accordance with the invention are now described. The connectors shown in FIGS. 17-20 and 31-33 are all suitable for use with the apparatus and methods described with respect to FIGS. 1-16 for providing an anastomosis between an aperture in a side wall of a graft conduit and an aperture in a side wall of a patient's body tissue conduit. The connectors of FIGS. 17-20 and 31-33 may be of similar sizes and cross-sections as connectors 100 and 1100, and the connectors may be constructed of the same material or materials as connectors 100 and 1100. The differences between the embodiments of the connectors shown in FIGS. 17-20 and 31-33 and connectors 100 and 1100 are made apparent in the description that follows.

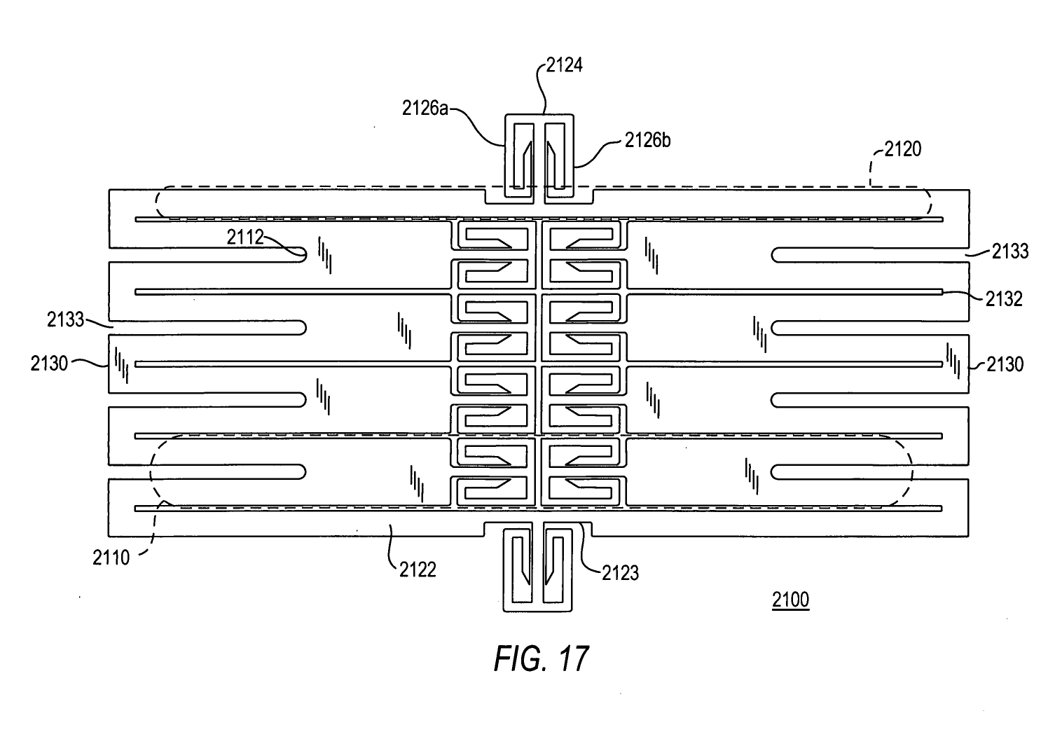

An illustrative embodiment of a self-expanding tubular connector 2100 in accordance with the invention is shown in FIGS. 17-20. Connector 2100 is substantially similar to connector 1100 (FIG. 10). However, a difference between the two connectors is that, while the proximal ends of fingers 2112 of each of the finger portion pairs 2110 and the proximal ends of fingers 2122 of each of the finger portion pairs 2120 of connector 2100 are indeed connected to common proximal body portions 2130 at junctions 2132, each finger portion pair 2110 may also have a substantially semi-annular slit 2133 running through proximal portion 2118 of connector 2100, instead of being solid at the proximal portion like each of portion pairs 1110 of connector 1100. This aspect of connector 2100 improves its axial and annular enlargeability. Slits 2133 in cooperation with junctions 2132 provide for greater expanded axial finger spacing distances 2129 between adjacent portion pairs 2110 (see, e.g., FIG. 20), and thus for a greater expanded axial opening distance between portion pairs 2120, than those that junctions 1132 can provide by themselves, as in connector 1100.

Another difference between connector 2100 and connector 1100 is that the tissue holding feature included at the distal ends of some or all of fingers 2112 and members 2123 of connector 2100 may include a “T-shaped” distal member 2124 that has a graft tissue hook 2126a and a body tissue hook 2126b for engaging tissue, instead of a distal member 1124 with only one free end portion 1126 as in connector 1100. This aspect of connector 2100 increases the perimeter of the anastomotic connection it is able to make between first conduit 200 (FIG. 4) and a second conduit. By bending graft tissue hooks 2126a proximally away from distal members 2124, perimeter 201 of the ostium created by connector 2100 through aperture 202 of the first conduit is defined by perimeter 203, and not by a perimeter smaller than aperture perimeter 203 as created by connector 1100.

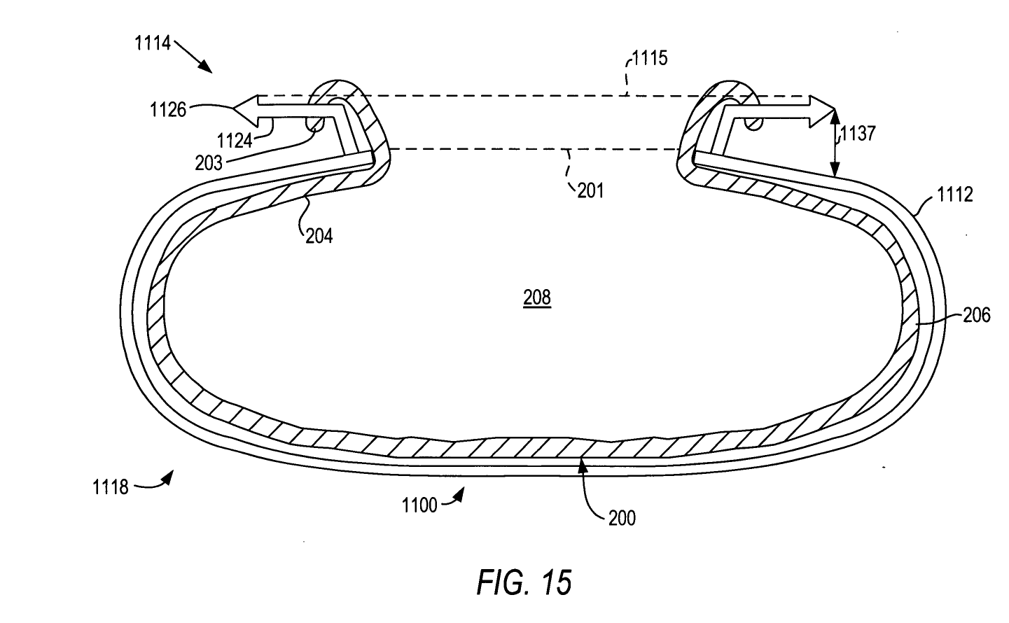

For attachment, first conduit 200 may be positioned within the hollow lumen of initial (i.e., non-expanded) tubular connector 2100, such that aperture 202 made in the side wall of first conduit 200 faces and is substantially parallel to initial distal perimeter 2111. As illustrated in FIG. 18, periphery 203 of aperture 202 is advanced in the direction of arrows 212 over proximally bent graft tissue hooks 2126a but under members 2124 and distally bent body tissue hooks 2126b. Along with this weaving of graft tissue over and under hooks 2126; the sharpened tip portions of hooks 2126a may engage with interior surface tissue 204 of conduit 200 in order to secure connector 2100 to the first conduit. Initial distal perimeter 2111 is defined by the ends of upwardly bent body tissue hooks 2126b. It is to be understood that in another embodiment, connector 2100 may be substantially annularly continuous but with the left and right edges of the structure shown in FIG. 17 separate from one another, although substantially close, while at least one end member 2123 allows the connector to remain a one-piece (unitary) structure.

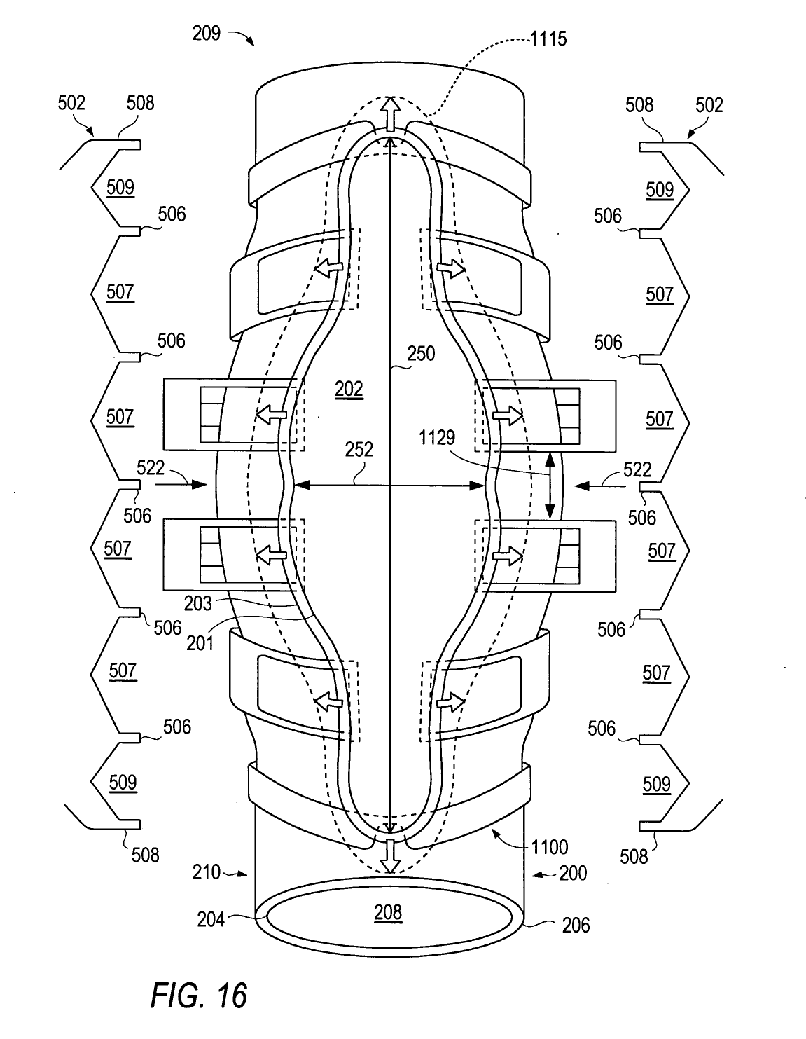

FIGS. 19 and 20 illustrate that once periphery 203 is advanced such that it is substantially held by graft tissue hooks 2126a, connector 2100 preferably enlarges to its expanded condition. Expansion of connector 2100 stretches periphery 203 tightly about graft tissue hooks 2126a such that periphery 203 substantially defines the ostium perimeter 201 created by the connector through aperture 202. Ostium perimeter 201 may generally be understood as having a substantially axial length 250 and a substantially trans-axial length 252. Perimeter 203 of first conduit 200 may be engaged with connector 2100 at the points where members 2124 meet graft tissue hooks 2126a. Expanded distal perimeter 2115 is defined by the tips of upwardly bent body tissue hooks 2126b. Spacing 2137 between the ends of hooks 2126a and 2126b of a tissue holding feature may vary depending on the wall thicknesses of the two conduits to be joined by connector 2100. This aspect of the invention will be described in greater detail herein.

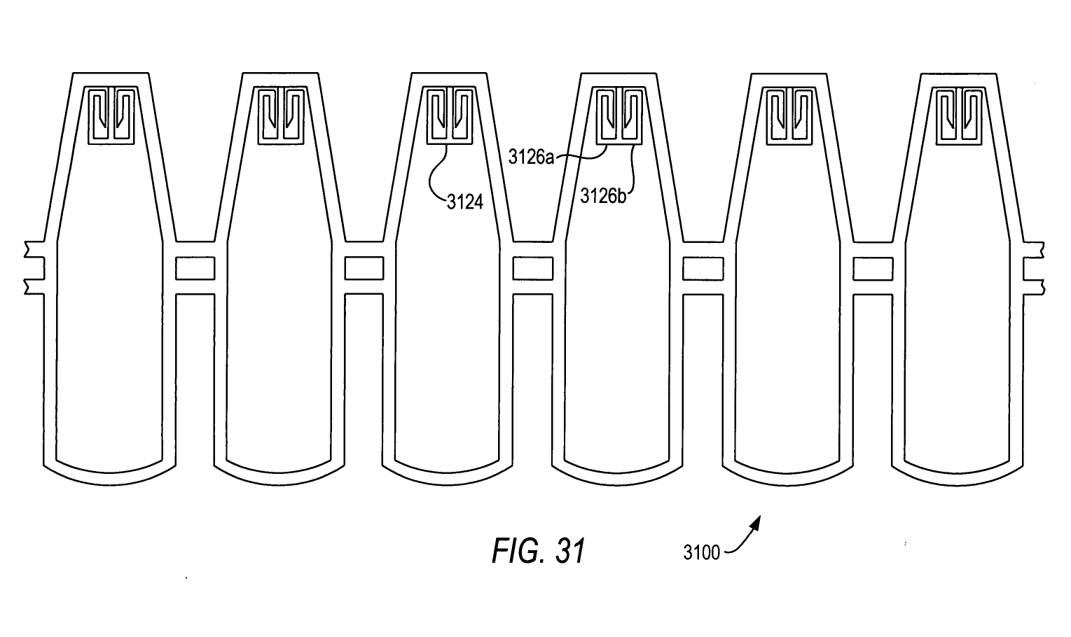

It is to be understood that the tissue holding features of connector 1100 could be implemented on connector 2100, for example, at end members 2123 instead of the “hook-type” shown, to hold a “bulge” of graft tissue which often exists at the ends of the ostium. As shown in FIG. 31 by connector 3100, it is also to be understood that the tissue holding features of connector 2100 could be implemented on the structure of cellular connector 100 and used in the same way without departing from the spirit and scope of the present invention. Furthermore, it is also to be understood that slits 2133 of connector 2100 could be implemented on the structure of connector 1100 and used in the same way without departing from the spirit and scope of the present invention.

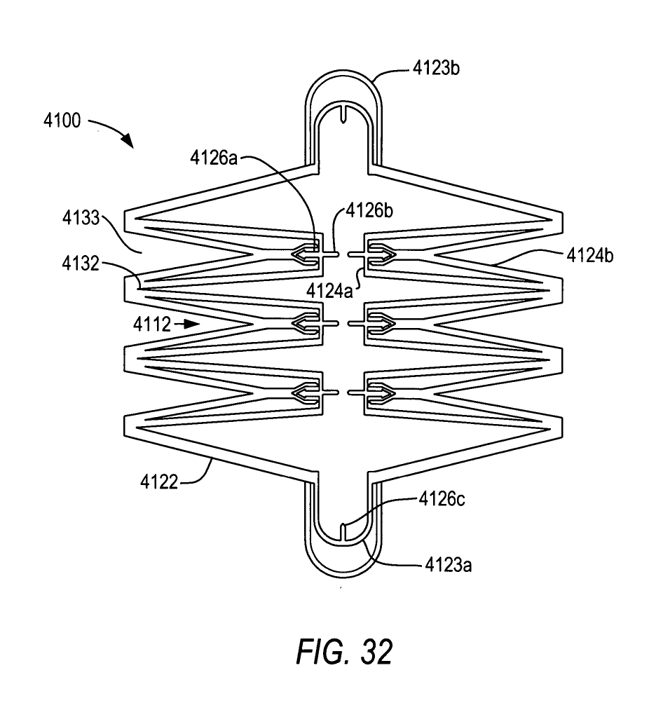

An illustrative embodiment of another first connector 4100 in accordance with the invention is shown in FIG. 32. Connector 4100 is substantially similar to connector 2100 (FIG. 17). However, a difference between the two connectors is that fingers 4112 of connector 4100 have a slightly different shape than fingers 2112 of connector 2100. For example, outer finger member pairs 4124a and inner finger member pairs 4124b run along substantially the whole length of fingers 4112 and slits 4133 starting at junctions 4132. Finger member pairs 4124 may expand axially and annularly like fingers 1112 of connector 1100 and fingers 2112 of connector 2100. Connector 4100 may include a graft tissue hook 4126a and a body tissue hook 4126b at the distal end of each outer finger member pair 4124a. Graft tissue hooks 4126a may expand or be bent proximally away from member pairs 4124a, and body tissue hooks 4126b may expand or be bent distally away from member pairs 4124b. Connector 4100 may also include an inner end member 4123a with a graft tissue hook 4126c and an outer end member 4123b at the junctions of end members 4122. Graft tissue hooks 4126c may also expand or be bent proximally away from inner end members 4123a, and outer end members 4123b may expand or be bent distally away from inner end members 4123a to define a distal perimeter for introduction into the lumen of a second conduit. This aspect of the invention will be described in greater detail herein.

First conduit 200 may be attached to connector 4100 in a way similar to how first conduit 200 is attached to connector 2100 (FIGS. 18-20), for example. However, periphery 203 of aperture 202 of first conduit 200 may be positioned under member pairs 4124 and end members 4123 so that graft tissue hooks 4126a and 4126c penetrate and pass through the side wall of conduit 200 from exterior surface 206 to interior surface 204, as a result of, for example, compressing the graft against the tips of graft tissue hooks 4126a and 4126c with tool 300 (FIGS. 7 and 14). Once periphery 203 is advanced such that it is substantially held by graft tissue hooks 4126a and 4126c, connector 4100 preferably enlarges to its expanded condition, thereby stretching periphery 203 such that it substantially defines ostium perimeter 201 created by connector 4100 through aperture 202, and such that inner finger member pairs 4124b rest on top of first conduit 200 against exterior wall 206.

Once first conduit 200 has been attached to a self-expanding exterior connector as described above, the connector may be deformed such that its distal perimeter is collapsed both axially and trans-axially for insertion into a bypass vessel (i.e., a body tissue conduit). The self-expanding exterior connector may be introduced and deployed after an aperture having a known and controlled perimeter is created in a side wall of the body tissue conduit.

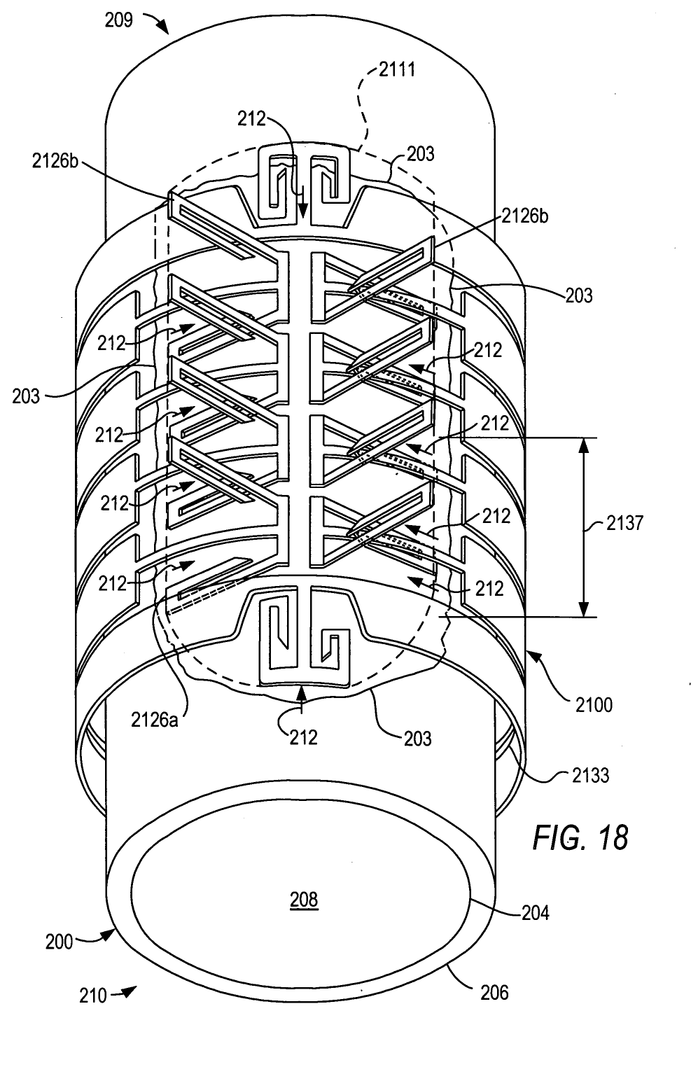

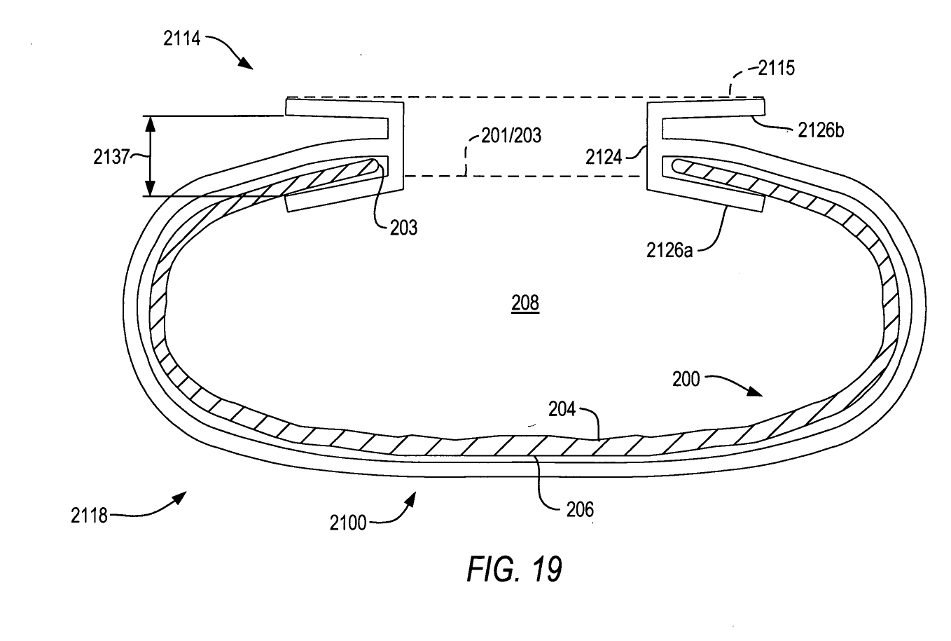

An illustrative tool 500 for deforming and delivering any of the above-described self-expanding exterior connectors (along with attached first conduit 200) to an operative site, and for making a hollow annular anastomotic connection between first conduit 200 and a second conduit, is shown in FIGS. 21-27. As described above, first conduit 200 is typically a graft conduit and may be a natural conduit, such as a saphenous vein graft (“SVG”), a synthetic conduit, or a combination thereof. The second conduit is typically a patient's natural body tissue conduit, such as a coronary artery.

As shown in FIGS. 21-27, delivery tool 500 includes first and second jaws 502 hinged at a pivot 504. At distal end 514 of tool 500, jaws 502 are spaced from one another by an opening 503, and each one of jaws 502 includes a plurality of teeth 506 spaced by openings 507. Furthermore, each one of jaws 502 may include first and second “molars” 508 flanking its plurality of teeth 506. Each one of molars 508 may be spaced from its adjacent tooth 506 by an opening 509. Each one of jaws 502 may also include a support 510 between its molars 508 and pivot 504. The number, size, and shape of teeth 506, molars 508, and supports 510 may vary depending on the geometry of conduit 200 and the geometry of the connector to which it is attached. Tool 500 may be manipulated manually (e.g., by a physician) to vary the size of opening 503, and, therefore, the size and shape of hollow 511 between pivot 504 and opening 503. Actuation of the delivery tool may be at the connector (i.e., at distal end 514, or at some location remote to the connector (i.e., at proximal end 512 outside of the chest cavity of the patient), and the actuation may be caused by direct force or may, for example, be hydraulically or cable driven. A catheter may be used in cooperation with proximal end 512 to provide manipulation of tool 500 at a distance from distal end 514.

For deformation of an expanded exterior connector of the invention that is attached to first conduit 200, jaws 502 may be positioned about the connector and lumen 208 of first conduit 200 such that opening 503 faces and is substantially parallel to the expanded distal perimeter of the connector, and such that at least the proximal portion of the connector is positioned within hollow 511. As illustrated in each of FIGS. 8, 16, and 20, tool 500 may be manipulated to advance each tooth 506 and molar 508 of jaws 502 in the direction of arrow 522, such that some or all of openings 507 and 509 each contains either a cell portion 112 of connector 100 (FIG. 8), a finger 1112 or a finger 1122 of connector 1100 (FIG. 16), or a finger 2112 or a finger 2122 of connector 2100 (FIG. 20) at a point just proximal to that element's tissue holding feature, thereby collapsing the connector axially and radially.

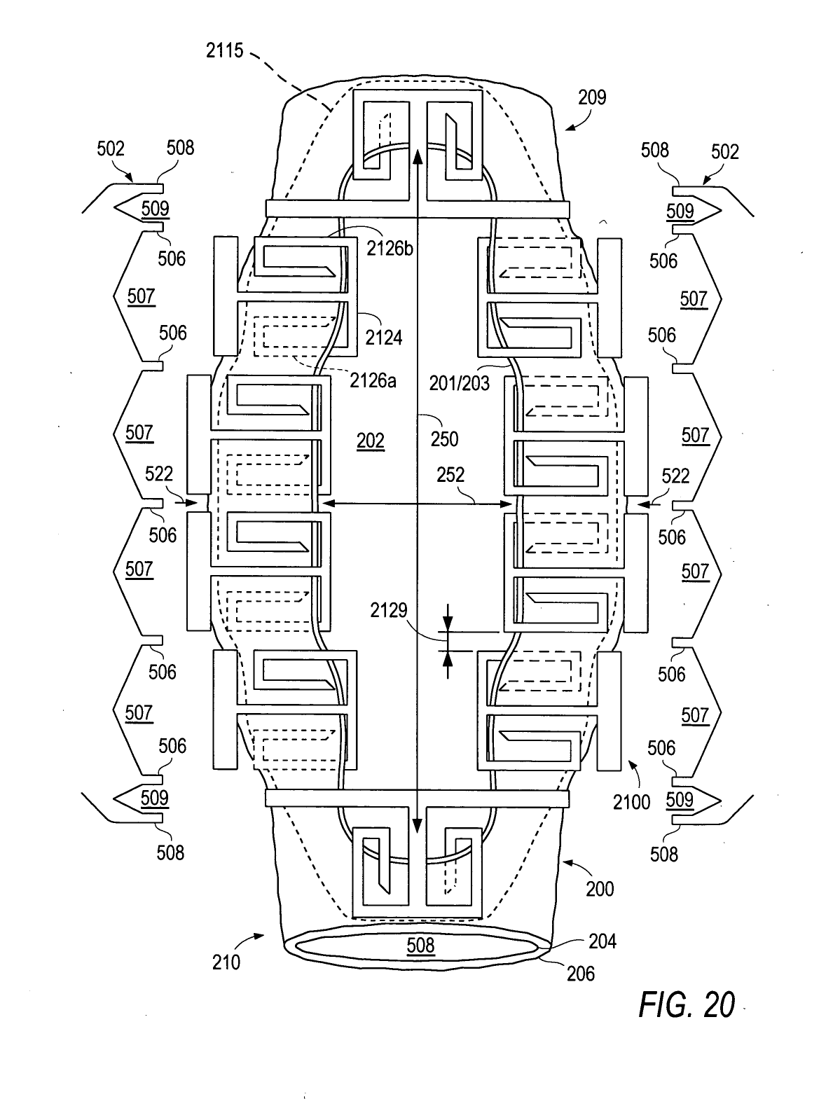

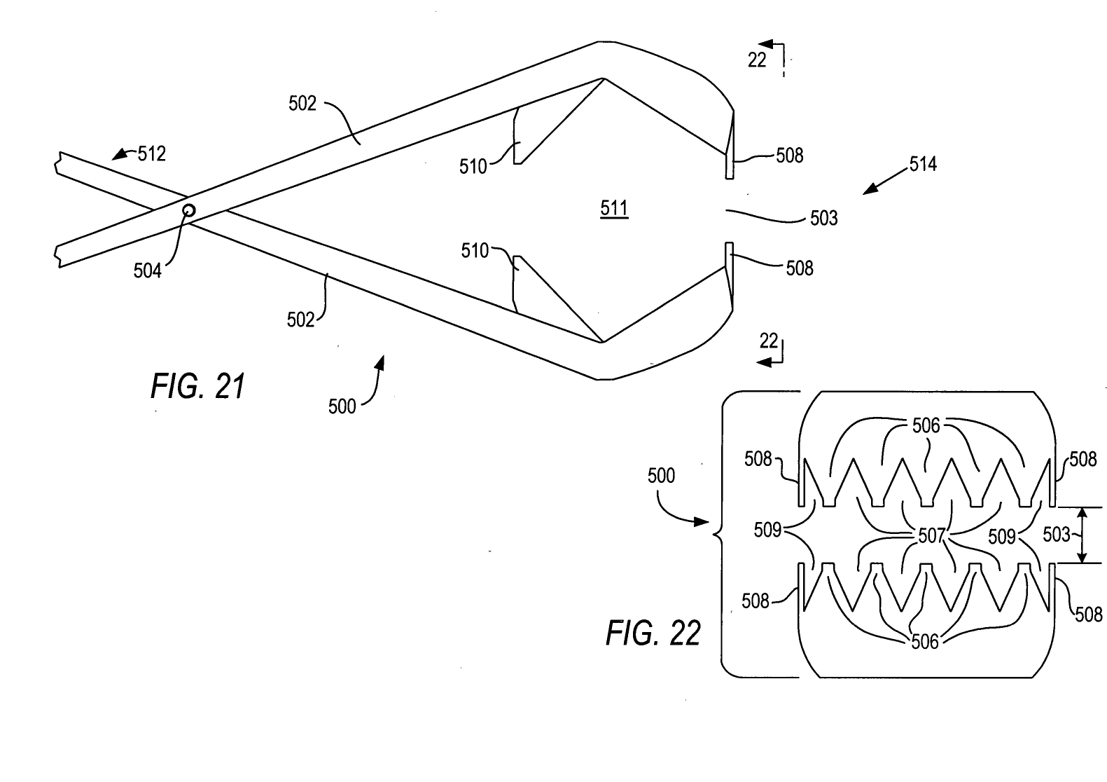

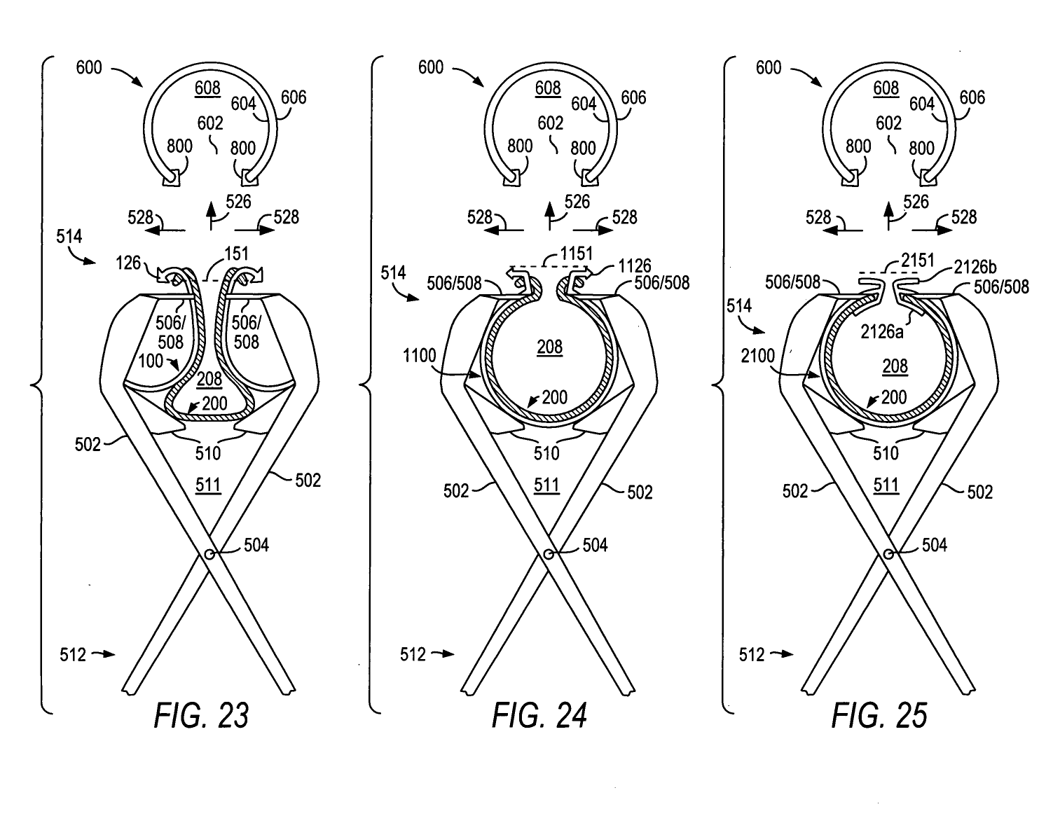

FIGS. 23-25 illustrate that teeth 506 and molars 508 are advanced to deform the connector until a collapsed distal perimeter is defined that is suitable for insertion into the second conduit. As shown in FIG. 23, delivery tool 500 has been manipulated to deform self-expanding cellular exterior connector 100 such that a collapsed distal perimeter 151 is defined outside of hollow 511 by the pointed tips of free end portions 126. Supports 510 may contribute to the deformation of connector 100 (i.e., proximal portion 118) and to the deformation of conduit 200. As shown in FIG. 24, delivery tool 500 has been manipulated to deform self-expanding tubular exterior connector 1100 such that a collapsed distal perimeter 1151 is defined outside of hollow 511 by the pointed tips of free end portions 1126. Supports 510 may contribute to the deformation of connector 1100 (i.e., proximal portion 1118). As shown in FIG. 25, delivery tool 500 has been manipulated to deform self-expanding tubular exterior connector 2100 such that a collapsed distal perimeter 2151 is defined outside of hollow 511 by the upwardly bent body tissue hooks 2126b. Supports 510 may contribute to the deformation and/or support of connector 2100 (i.e., proximal portion 2118).

FIGS. 23-25 also illustrate a typical use of delivery tool 500 to deliver a deformed exterior connector and first conduit 200 to an operative site for making a hollow annular anastomotic connection to an aperture in a side wall of a second conduit, typically a patient's tubular body tissue conduit (e.g., a coronary artery requiring a bypass graft).

An aperture 602 may be made in second conduit 600 in any manner described hereinabove with respect to making aperture 202 in first conduit 200. Aperture 602 is typically made downstream from an occlusion or lesion in lumen 608 of second conduit 600.

In some embodiments of the present invention, a connector may be installed in an incision in a bypass vessel (e.g., aperture 602 in second conduit 600). The assembly that includes another connector and a graft conduit (e.g., connectors 100, 2100, 3100, and 4100, and first conduit 200) may be coupled to the connector in the body tissue conduit. For simplicity, the connector installed in the aperture in the body tissue conduit is referred to herein as the “second connector,” and the connector assembled with the graft tissue conduit is referred to hereinafter as the “first connector.” One or both of the graft tissue conduit and the body tissue conduit may be small diameter vessels. The aperture into which the second connector is inserted may be created using the apparatus and methods described hereinabove for creating an aperture having a controlled, predetermined length.

By inserting a second connector into the aperture in the body tissue conduit, a physician may first create and inspect connector assemblies for each vessel (e.g., an assembly that includes the first connector and the graft tissue conduit, and an assembly that includes the second connector and the body tissue conduit), and then couple the two assemblies to one another. Although the second connector will continue to be referred to herein as a “connector,” in actuality the second connector may be any structure that serves to hold the incision in the body tissue conduit open and to keep the tissue layers of the vessel together. The second connector may be similar to, for example, an internal traction device that remains permanently in place. The second connector is mounted in the body tissue conduit without another vessel (e.g., a graft tissue conduit) attached to it.

Second connector 800 may be installed in aperture 602 of second conduit 600 prior to attachment of the first connector thereto. Second connector 800 may be a hollow annular connector used to hold open aperture 602 and to hold together the wall tissue of second conduit 600 about the aperture. Examples of apparatus and methods for installing a second connector in an incision in a second conduit (e.g., a target vessel) are described, for example, in concurrently-filed, commonly-assigned U.S. patent application Ser. No.______ (Atty. Docket No. 293/052), filed Oct. 3, 2003, which is incorporated by reference hereinabove, commonly-assigned U.S. patent application Ser. No. 10/158,436, and Swanson et al. U.S. Pat. No. 6,602,263, which are hereby incorporated by reference herein in their entireties.

For deployment of a self-expanding exterior connector of the invention that is attached to first conduit 200, tool 500 may be manipulated to advance the collapsed distal perimeter of the connector in the direction of arrow 526 through aperture 602 of second conduit 600, such that at least the elements of the connector which define its collapsed distal perimeter are positioned within lumen 608 of the second conduit while tool 500 remains substantially external to lumen 608. Then, tool 500 may be manipulated to increase the size of opening 503 by advancing the distal end of each jaw 502 in the direction of arrow 528, such that the connector may expand to its expanded condition and tool 500 may be disemployed.

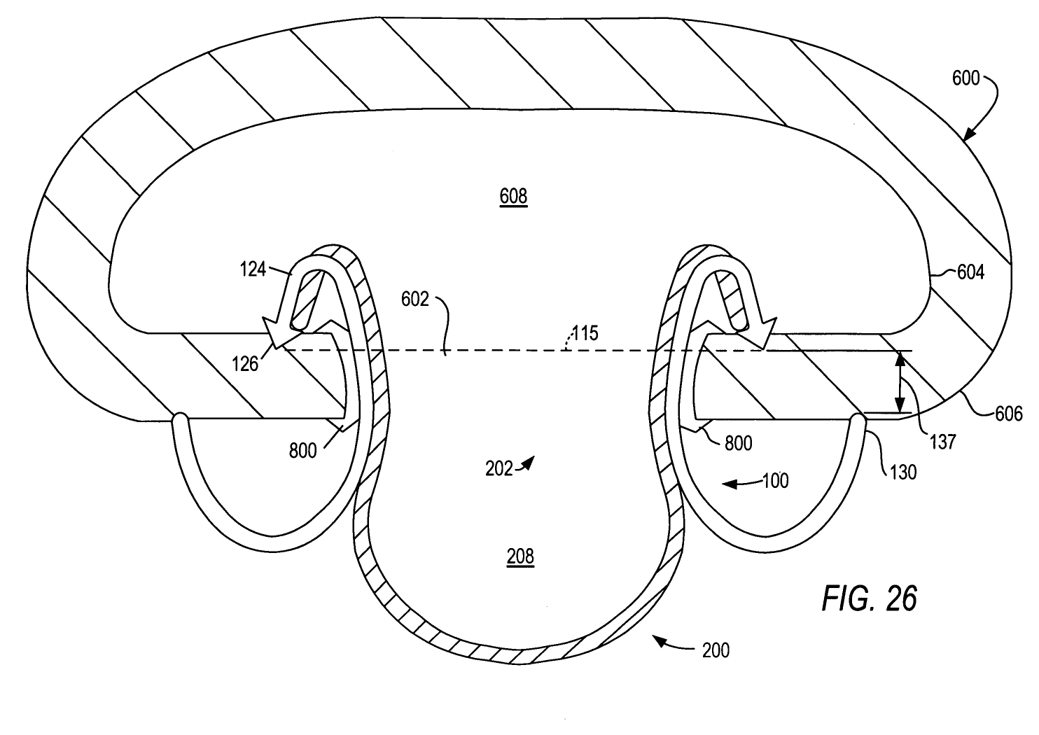

FIGS. 26-28 illustrate that the elements of the connector which define its collapsed distal perimeter in the deformed condition are introduced into and deployed within lumen 608 of second conduit 600 and tool 500 is disemployed. As shown in FIG. 26, cellular exterior connector 100 has expanded such that expanded distal perimeter 115 (i.e., the pointed tips of free end portions 126), portions of distal members 124, and portions of first graft conduit 200 including periphery 203 are positioned within lumen 608 and may be engaged with interior surface 604 of second body tissue conduit 600 about aperture 602, while medial portion 116 of connector 100 may be in contact with second connector 800 external to lumen 208 of first conduit 200 and lumen 608 of second conduit 600. In a preferred embodiment, the tips of members 130 are positioned outside of lumen 608 and may be engaged with exterior surface 606 of second conduit 600 about aperture 602. Preferably, after expansion of the connector, axial spacing 137 is equal to (if not slightly less than) the tissue thickness of second conduit 600 (i.e., the distance between surfaces 604 and 606) in order to create a fluid-tight anastomotic connection between conduit 200 and conduit 600.

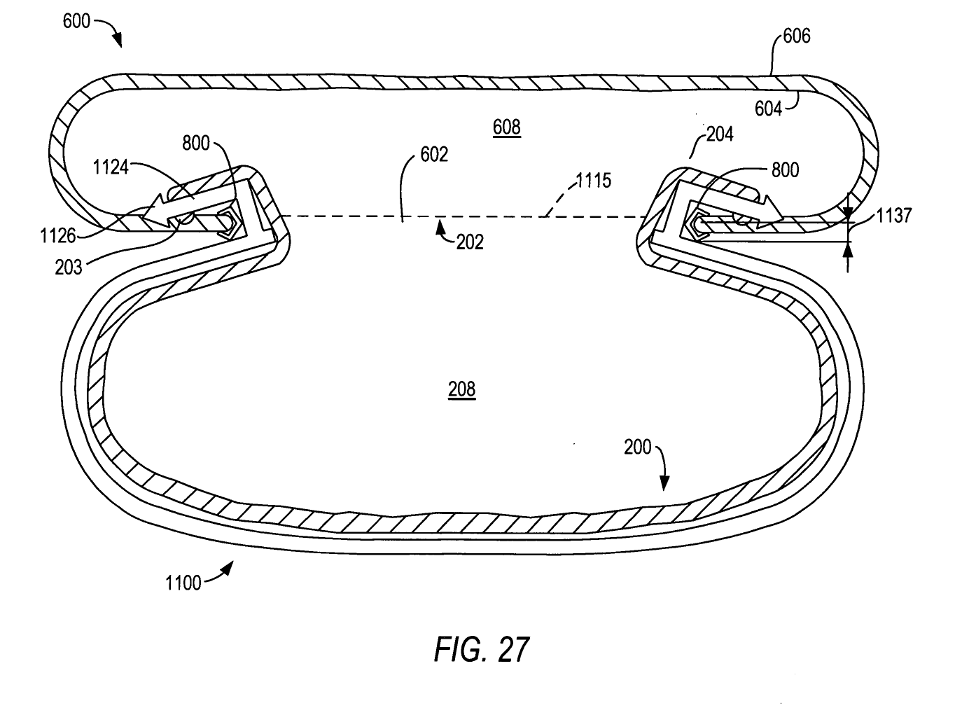

Similarly, as shown in FIG. 27, tubular exterior connector 1100 has expanded such that expanded distal perimeter 1115 (i.e., the pointed tips of free end portions 1126), portions of distal members 1124, and portions of first graft conduit 200 including periphery 203, are positioned within lumen 608 and may be engaged with interior surface 604 of second body tissue conduit 600 about aperture 602, while medial portion 1116 of connector 1100 may be in contact with second connector 800 external to lumen 208 of first conduit 200 and lumen 608 of second conduit 600. In a preferred embodiment, the portion of fingers 1112 and 1122 just proximal to their tissue holding features are positioned outside of lumen 608 and may be engaged with exterior surface 606 of second conduit 600 about aperture 602. Preferably, after expansion of the connector, axial spacing 1137 is equal to (if not slightly less than) the tissue thickness of second conduit 600 in order to create a fluid-tight anastomotic connection between conduit 200 and conduit 600.

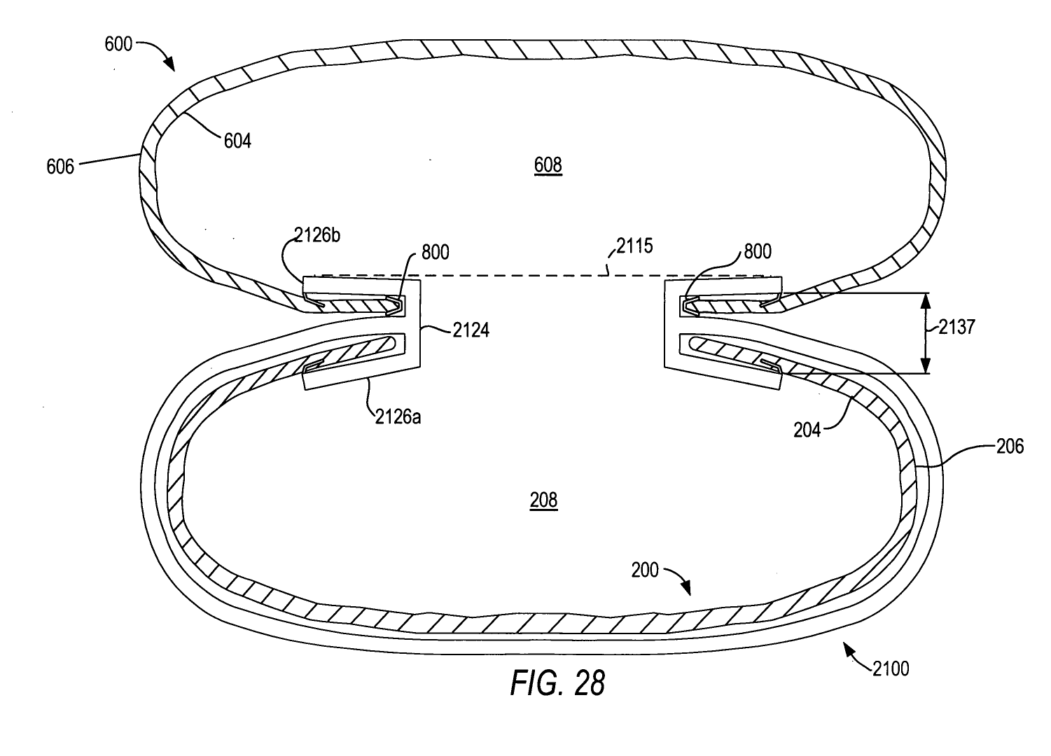

Finally, as shown in FIG. 28, tubular exterior connector 2100 has expanded such that expanded distal perimeter 2115 (i.e., the ends of body tissue hooks 2126b) and portions of distal members 2124 are positioned within lumen 608 and may be engaged with interior surface 604 of second body tissue conduit 600 about aperture 602, while other portions of distal members 2124 may be in contact with second connector 800. However, still other portions of distal members 2124, all or substantially all of graft conduit 200, and graft tissue hooks 2126a are positioned outside of lumen 608 and may be engaged with exterior surface 606 of second conduit 600 about aperture 602. Preferably, after expansion of the connector, axial spacing 2137 is equal to (if not slightly less than) the tissue thickness of second conduit 600 plus the tissue thickness of first conduit 200 (i.e., the distance between surfaces 204 and 206) in order to create a fluid-tight anastomotic connection between conduit 200 and conduit 600 about apertures 202 and 602.

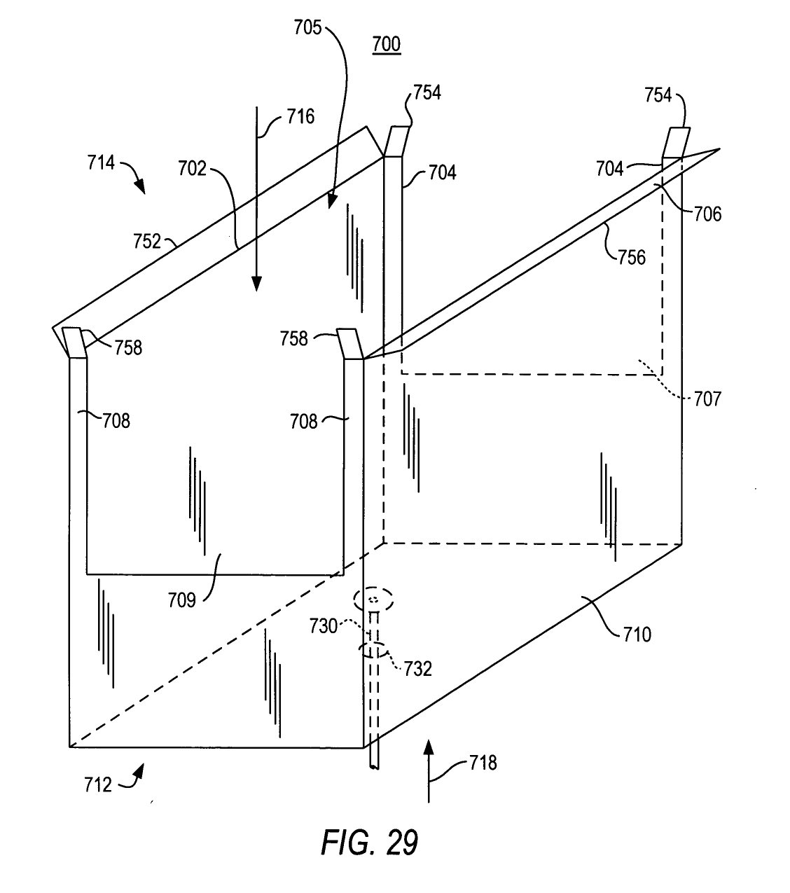

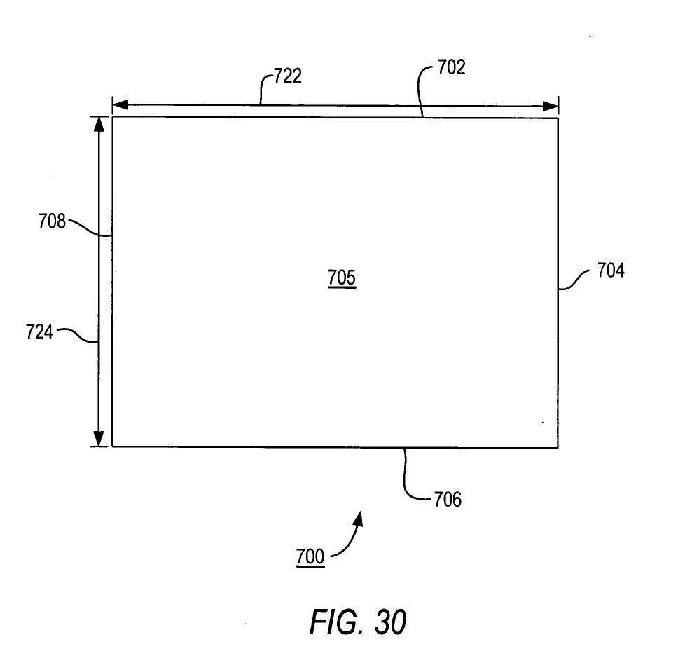

An alternative embodiment of a delivery tool in accordance with the invention is now described. The delivery tool shown in FIGS. 29 and 30 is suitable for use with the apparatus and methods described with respect to FIGS. 1-20 and 31-33 for deforming and delivering a self-expanding exterior connector and first conduit to an operative site, and for making a hollow annular anastomotic connection between the first conduit and the second conduit. Delivery tool 700 is a rigid and hollow substantially cube-like structure defined by side walls 702, 704, 706, and 708. Tool 700 may also include a bottom wall 710 at its proximal end 712. Distal end 714 of tool 700 provides an opening 705 which may be of any suitable size and shape for deforming the connector attached to the first conduit. In a preferred embodiment, opening 705 is rectangularly shaped with a length 722 and a width 724.

An expanded connector of the invention that is attached to first conduit 200 may then be deformed by being loaded into tool 700 through opening 705 in the direction of arrow 716. By inserting the proximal end of the connector down into tool 700 through opening 705, the connector is deformed such that the collapsed distal perimeter is defined external to tool 700 distally from end 714. The connector may first be deformed manually by a physician or by other tools such that its proximal end can be passed through opening 705 and down into the structure of tool 700. Alternatively, splays 752, 754, 756, and 758 may be provided distally to opening 705 in order to deform the expanded connector and guide it through opening 705. Side openings 707 and 709 may be made in side walls 704 and 708, respectively, such that the length of the first conduit (i.e., from first end 209 to second end 210) attached to the connector may extend through the structure of tool 700 once the connector is deformed and loaded into tool 700. Once tool 700 contains at least the proximal end of the connector such that a collapsed distal perimeter is defined thereby, tool 700 may deliver the connector and first conduit to an operative site for making an anastomotic connection to an aperture in a side wall of a second conduit as described above with respect to tool 500. Then, tool 700 may release the connector by advancing rod 730 in the direction of arrow 718 up through hole 732 in bottom wall 710, for example, thereby pushing the proximal end of the connector up through opening 705.

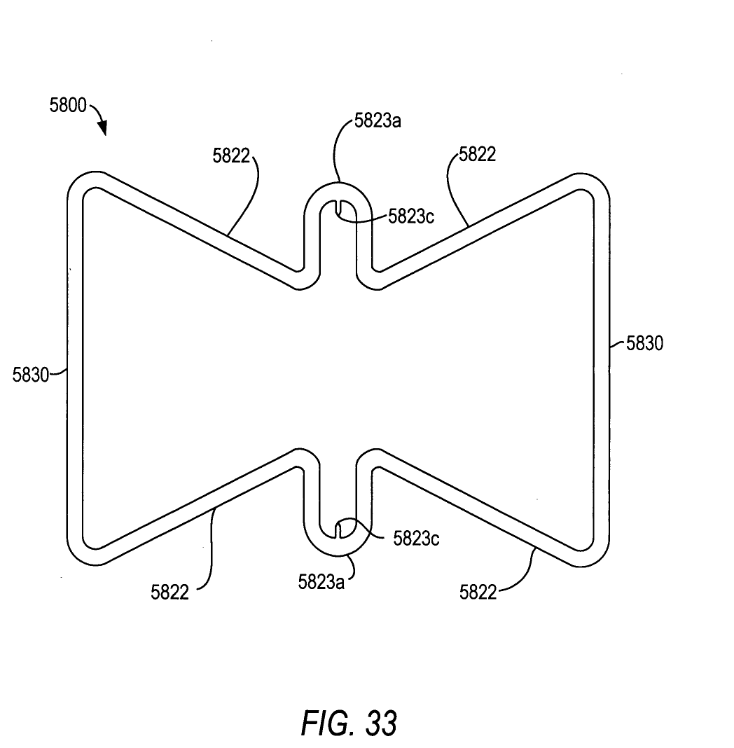

An illustrative embodiment of a self-expanding tubular exterior second connector 5800 in accordance with the invention is shown in planar development in FIG. 33. Connector 5800 may be installed in aperture 602 of second connector 600 prior to attachment of the first connector thereto, substantially similarly to second connector 800 (FIGS. 23-28). Second connector 5800 may include an inner end member 5823a with a body tissue hook 5823c at the junctions of end members 5822, and end members 5822 may meet at distal members 5830. Second connector 5800 may be attached to second conduit 600 by passing inner end members 5823a through aperture 602 into lumen 608 of second connector 600.

Once inner end members 5823a are advanced such that they engage interior wall 604 of second conduit 600 about aperture 602 and such that end members 5822 engage exterior wall 606 of second conduit 600 about aperture 602, connector 5800 preferably expands, thereby holding open aperture 602 and holding together the wall tissue of second conduit 600 about aperture 602 between interior wall 604 and exterior wall 606. When connector 5800 expands, distal members 5830 may contact exterior wall 606 of conduit 600 at its distal side opposite aperture 602. Each distal member 5830 may be easily deformed proximally away from exterior wall 606 of conduit 600, thereby varying the size of aperture 602 held open by the structure of second connector 5800, such that distal portions of a first connector may be advanced therethrough to create an anastomosis, as described herein above.

Thus it is seen that a self-expanding exterior connector for creating anastomoses to small-diameter vessels and methods of use have been provided. One skilled in the art will appreciate that the present invention can be practiced by other than the described embodiments, which are presented for purposes of illustration and not of limitation, and the present invention is limited only by the claims which follow.

What is claimed is:

1. A method of producing a hollow anastomotic connection between a first aperture in a side wall defined by first and second ends of a graft tissue conduit and a second aperture in a side wall defined by first and second ends of a body tissue conduit in a patient using a hollow annular connector having a first portion and a second portion, wherein the first portion has a plurality of first members, wherein a distal perimeter is defined by at least a first group of the plurality of first members, and wherein a first spacing is defined between at least the first group of first members and a third portion of the connector proximal to the first group of first members, comprising:
introducing the graft tissue conduit into the hollow connector so that tissue of the graft tissue conduit about the first aperture is retained by at least a second group of the plurality of first members and so that at least the first end of the graft tissue conduit extends out of the hollow connector via the second portion;
deforming the connector so that the distal perimeter collapses;
approximating the first and second apertures so that at least the first group of first members of the connector extends into the body tissue conduit via the second aperture; and
reforming the connector so that the distal perimeter expands, so that the first spacing decreases, and so that at least the first group of first members and the third portion of the connector press together the side walls of the body tissue conduit and the graft tissue conduit annularly around the first and second apertures.

2. The method defined in claim 1, wherein the first group of first members at least includes the second group of first members so that tissue of the graft tissue conduit extends from within the lumen of the body tissue conduit to outside of the body tissue conduit, and so that body fluid of the patient can flow between the lumen of the graft tissue conduit and the lumen of the body tissue conduit via the connection.

3. The method defined in claim 1, wherein the deforming comprises:
annularly compressing the connector.

4. The method defined in claim 1 further comprising:
before the deforming, providing a delivery tool configured to retain a retainable portion of the connector proximal to the first group of first members, and
wherein the deforming the connector comprises retaining the retainable portion of the connector with the delivery tool.

5. The method defined in claim 4, wherein the reforming the connector further comprises:
releasing the retainable portion of the connector from the delivery tool.

6. A method of producing a hollow anastomotic connection between a first aperture in a side wall defined by first and second ends of a graft tissue conduit and a second aperture in a side wall defined by first and second ends of a body tissue conduit in a patient using a hollow annular connector having a distal axial portion, a proximal axial portion, and a medial portion therebetween, wherein the distal axial portion has a plurality of first members and the proximal axial portion has a plurality of second members, wherein both a distal perimeter defined by at least a first group of the plurality of first members and a proximal perimeter defined by the second members are larger than a medial perimeter of the medial portion, and wherein an axial spacing is defined between at least the first group of first members and the second members, comprising:
introducing the side wall of the graft tissue conduit into the hollow annular connector so that tissue of the graft tissue conduit about the first aperture is retained by at least a second group of the plurality of first members and so that at least the first end of the graft tissue conduit extends out of the hollow connector via the proximal portion;
deforming the connector so that the distal perimeter collapses;
approximating the first and second apertures so that at least the first group of first members of the connector extends into the body tissue conduit via the second aperture; and
reforming the connector so that the distal perimeter expands, so that the axial spacing decreases, and so that at least the first group of first members and the second members press together the side walls of the body tissue conduit and the graft tissue conduit annularly around the first and second apertures.

7. The method defined in claim 6, wherein the first group of first members at least includes the second group of first members so that tissue of the graft tissue conduit extends from within the lumen of the body tissue conduit to outside of the body tissue conduit, and so that body fluid of the patient can flow between the lumen of the graft tissue conduit and the lumen of the body tissue conduit via the connection.

8. The method defined in claim 6, wherein the deforming comprises:
annularly compressing the connector.

9. The method defined in claim 6 further comprising:
before the deforming, providing a delivery tool configured to retain a retainable portion of the connector proximal to the first group of first members, and
wherein the deforming the connector comprises retaining the retainable portion of the connector with the delivery tool.

10. The method defined in claim 9, wherein the reforming the connector further comprises:
releasing the retainable portion of the connector from the delivery tool.

11. A method of producing a hollow anastomotic connection between a first aperture in a side wall defined by first and second ends of a graft tissue conduit and a second aperture in a side wall defined by first and second ends of a body tissue conduit in a patient using a hollow annular connector having a first axial end portion, a second axial end portion, and a medial portion therebetween, wherein the medial portion has a plurality of first members about an opening, wherein a distal perimeter is defined by at least a first group of the plurality of first members, and wherein a first spacing is defined between at least the first group of first members and the medial portion, comprising:
introducing the graft tissue conduit into the hollow connector so that tissue of the graft tissue conduit about the first aperture is retained by at least a second group of the plurality of first members and so that the first and second ends of the graft tissue conduit extend out of the hollow connector via the first and second axial end portions, respectively;
deforming the connector so that the distal perimeter collapses;
approximating the first and second apertures so that at least the first group of first members of the connector extends into the body tissue conduit via the second aperture; and
reforming the connector so that the distal perimeter expands, so that the first spacing decreases, and so that at least the first group of first members and the medial portion press together the side walls of the body tissue conduit and the graft tissue conduit annularly around the first and second apertures.

12. The method defined in claim 11, wherein the first group of first members at least includes the second group of first members so that tissue of the graft tissue conduit extends from within the lumen of the body tissue conduit to outside of the body tissue conduit, and so that body fluid of the patient can flow between the lumen of the graft tissue conduit and the lumen of the body tissue conduit via the connection.

13. The method defined in claim 11, wherein the deforming comprises:
annularly compressing the connector.

14. The method defined in claim 11, wherein the deforming comprises:
axially compressing the connector.

15. The method defined in claim 11 further comprising:
before the deforming, providing a delivery tool configured to retain a retainable portion of the connector proximal to the first group of first members, and
wherein the deforming the connector comprises retaining the retainable portion of the connector with the delivery tool.

16. The method defined in claim 15, wherein the reforming the connector further comprises:
releasing the retainable portion of the connector from the delivery tool.

17. A connector for use in making a hollow anastomotic connection between a first aperture in a side wall defined by first and second ends of a tubular graft tissue conduit and a second aperture in a side wall defined by first and second ends of a tubular body tissue conduit in a patient, the connector comprising:
a structure that is substantially annularly continuous but annularly enlargeable about its longitudinal axis, the structure including:
a first portion, wherein the first portion includes a plurality of first members extending away from the structure, wherein a distal perimeter is defined by at least a first group of the plurality of first members configured to engage the interior wall of the body tissue conduit about the second aperture, and wherein a second group of the plurality of first members is configured to engage the graft tissue conduit about the first aperture; and
a second portion proximal to the first group of first members, wherein a first spacing is defined between at least the first group of first members and the second portion, and wherein the structure is configured to expand from a deformed configuration having a collapsed distal perimeter to an expanded configuration having an expanded distal perimeter.

18. The connector defined in claim 17, wherein the first and second groups of first members are substantially radially aligned with respect to a common axis.

19. The connector defined in claim 18, wherein the first members of the first group extend distally away from the first portion of the structure and wherein the first members of the second group extend proximally away from the first portion of the structure.

20. The connector defined in claim 19, wherein each of the first members of the first group has a hook with a sharp end portion for engaging tissue of the interior wall of the body tissue conduit.

21. The connector defined in claim 20, wherein each of the first members of the second group has a hook with a sharp end portion for engaging tissue of the interior wall of the graft tissue conduit.

22. The connector defined in claim 18, wherein the first group of first members at least includes the second group of first members so that the first group of first members is configured to engage both the graft tissue conduit about the first aperture and the interior wall of the body tissue conduit about the second aperture, so that tissue of the graft tissue conduit can extend from within the lumen the body tissue conduit to outside of the body tissue conduit, and so that body fluid of the patient can flow between the lumen of the graft tissue conduit and the lumen of the body tissue conduit via the connection.

23. The connector defined in claim 22, wherein each of the first members of the first group has a barbed end portion.

24. A connector for use in making a hollow anastomotic connection between a first aperture in a side wall defined by first and second ends of a tubular graft tissue conduit and a second aperture in a side wall defined by first and second ends of a tubular body tissue conduit in a patient, the connector comprising:
a hollow structure that is substantially annularly continuous but annularly enlargeable about its longitudinal axis and configured for disposition substantially perpendicular to the longitudinal axis of the tubular graft conduit and the tubular body conduit, the structure including:
a distal axial portion, wherein a plurality of first members extend away from the distal axial portion in an annular array that is substantially concentric with the structure, wherein a distal perimeter is defined by at least a first group of the plurality of first members configured to engage the interior wall of the body tissue conduit about the second aperture, and wherein a second group of the plurality of first members is configured to engage the graft tissue conduit about the first aperture;
a proximal axial portion, wherein a proximal perimeter is defined by a plurality of second members of the proximal axial portion configured to engage the exterior wall of the body tissue conduit about the second aperture; and
a medial axial portion between the distal axial portion and the proximal axial portion, wherein an axial spacing is defined between at least the first group of first members and the plurality of second members, and wherein the structure is configured to expand from a deformed configuration having a collapsed distal perimeter and a first axial spacing to an expanded configuration having an expanded distal perimeter and a second axial spacing.

25. The connector defined in claim 24, wherein the medial axial portion is configured to extend in a first direction along the exterior of the graft tissue conduit about the first aperture substantially perpendicular to the longitudinal axis of the graft tissue conduit.

26. The connector defined in claim 24, wherein the distal axial portion is configured to receive tissue of the graft tissue conduit about the first aperture extending up through the hollow interior of the structure in a direction substantially perpendicular to the longitudinal axis of the graft tissue conduit.

27. The connector defined in claim 24, wherein the collapsed distal perimeter is smaller than the perimeter of the second aperture.

28. The connector defined in claim 24, wherein the second axial spacing is smaller than the first axial spacing.

29. The connector defined in claim 24, wherein at least the first group of first members and the plurality of second members are configured to resiliently press the graft tissue conduit and the body tissue conduit into annular contact with one another annularly around the first and second apertures.

30. The connector defined in claim 24, wherein the second axial spacing is substantially equal to the sum of the wall thickness of the graft tissue conduit and the wall thickness of the body tissue conduit.

31. The connector defined in claim 24, wherein the first and second groups of first members are substantially radially aligned with respect to the longitudinal axis of the structure.

32. The connector defined in claim 31, wherein the first members of the first group extend distally away from the proximal axial portion of the structure and wherein the first members of the second group extend proximally toward the proximal axial portion of the structure.

33. The connector defined in claim 32, wherein each of the first members of the first group has a hook with a sharp end portion for engaging tissue of the interior wall of the body tissue conduit.

34. The connector defined in claim 32, wherein each of the first members of the second group has a hook with a sharp end portion for engaging tissue of the interior wall of the graft tissue conduit.

35. The connector defined in claim 31, wherein the first group of first members at least includes the second group of first members so that the first group of first members is configured to engage both the graft tissue conduit about the first aperture and the interior wall of the body tissue conduit about the second aperture, so that tissue of the graft tissue conduit can extend from within the lumen the body tissue conduit to outside of the body tissue conduit, and so that body fluid of the patient can flow between the lumen of the graft tissue conduit and the lumen of the body tissue conduit via the connection.

36. The connector defined in claim 35, wherein each of the first members of the first group has a barbed end portion for engaginq tissue of the graft tissue conduit and tissue of the interior wall of the body tissue conduit.

37. A connector for use in making a hollow anastomotic connection between a first aperture in a side wall defined by first and second ends of a tubular graft tissue conduit and a second aperture in a side wall defined by first and second ends of a tubular body tissue conduit in a patient, the connector comprising:
a hollow structure that is substantially annularly continuous but enlargeable both annularly about and axially along its longitudinal axis, and configured for disposition substantially coincident with the longitudinal axis of the graft body conduit and substantially parallel to the longitudinal axis of the tubular body conduit, the structure including:
a first axial end portion;
a second axial end portion; and
a medial portion between the first and second axial end portions, wherein the medial portion includes a plurality of first members extending away from the structure and defining an opening in the medial portion, wherein a distal perimeter is defined by at least a first group of the plurality of first members configured to engage the interior wall of the body tissue conduit about the second aperture, wherein a second group of the plurality of first members is configured to engage the graft tissue conduit about the first aperture, wherein a medial spacing is defined between at least the first group of first members and a proximal portion of the medial portion proximal to the first group of first members, and wherein the structure is configured to expand from a deformed configuration having a collapsed distal perimeter and a first medial spacing to an expanded configuration having an expanded distal perimeter and a second medial spacing.

38. The connector defined in claim 37, wherein the first axial end portion is configured to extend in a first direction along the exterior of the graft tissue conduit from the first aperture, and wherein the second axial end portion is configured to extend in a second direction substantially opposite the first direction along the exterior of the graft tissue conduit from the first aperture.

39. The connector defined in claim 38, wherein each of the first and second directions is substantially parallel to the longitudinal axis of the graft tissue conduit.

40. The connector defined in claim 38, wherein each axial end portion is configured to extend only part way around a circumference of the exterior of the graft tissue conduit.

41. The connector defined in claim 38, wherein each axial end portion is configured to extend all the way around a circumference of the exterior of the graft tissue conduit.

42. The connector defined in claim 37, wherein the medial portion is configured to receive tissue of the graft tissue conduit about the first aperture extending up through the opening in the medial portion in a direction substantially perpendicular to the longitudinal axis of the graft tissue conduit.

43. The connector defined in claim 37, wherein the collapsed distal perimeter is smaller than the perimeter of the second aperture.

44. The connector defined in claim 37, wherein the second medial spacing is smaller than the first medial spacing.

45. The connector defined in claim 37, wherein at least the first group of first members and the proximal portion of the medial portion proximal to the first group of first members are configured to resiliently press the graft tissue conduit and the body tissue conduit into annular contact with one another annularly around the first and second apertures.

46. The connector defined in claim 37, wherein the second medial spacing is substantially equal to the sum of the wall thickness of the graft tissue conduit and the wall thickness of the body tissue conduit.

47. The connector defined in claim 37, wherein the first and second groups of first members are substantially radially aligned with respect to an ostium axis of the opening that is substantially perpendicular to the longitudinal axis of the structure.

48. The connector defined in claim 47, wherein the first members of the first group extend distally away from the structure and wherein the first members of the second group extend proximally away from the structure.

49. The connector defined in claim 48, wherein each of the first members of the first group has a hook with a sharp end portion for engaging tissue of the interior wall of the body tissue conduit.

50. The connector defined in claim 48, wherein each of the first members of the second group has a hook with a sharp end portion for engaging tissue of the interior wall of the graft tissue conduit.

51. The connector defined in claim 50, wherein the proximal portion of the medial portion includes the second group of first members.

52. The connector defined in claim 47, wherein the first group of first members at least includes the second group of first members so that the first group of first members is configured to engage both the graft tissue conduit about the first aperture and the interior wall of the body tissue conduit about the second aperture, so that tissue of the graft tissue conduit can extend from within the lumen the body tissue conduit to outside of the body tissue conduit, and so that body fluid of the patient can flow between the lumen of the graft tissue conduit and the lumen of the body tissue conduit via the connection.

53. The connector defined in claim 52, wherein each of the first members of the first group has a barbed end portion for engaging tissue of the graft tissue conduit and tissue of the interior wall of the body tissue conduit.

54. Apparatus for producing a hollow anastomotic connection between a first aperture in a side wall defined by first and second ends of a graft tissue conduit and a second aperture in a side wall defined by first and second ends of a body tissue conduit in a patient, comprising:
a connector having a structure that is substantially annularly continuous but annularly enlargeable about its longitudinal axis, the structure including:
a first portion, wherein the first portion includes a plurality of first members extending away from the structure, wherein a distal perimeter is defined by at least a first group of the plurality of first members configured to engage the interior wall of the body tissue conduit about the second aperture, and wherein a second group of the plurality of first members is configured to engage the graft tissue conduit about the first aperture; and
a second portion proximal to the first group of first members, wherein a first spacing is defined between at least the first group of first members and the second portion, and wherein the structure is configured to expand from a deformed configuration having a collapsed distal perimeter to an expanded configuration having an expanded distal perimeter; and
a delivery tool having a first configuration and a second configuration, wherein the first configuration is configured for retaining a retainable portion of the connector proximal to the first group of first members to deform the connector structure from the expanded configuration to the deformed configuration and to advance the collapsed distal perimeter of the connector into the lumen the body tissue conduit via the second aperture, and wherein the second configuration is configured for releasing the retainable portion of the connector to reform the connector structure from the deformed configuration to the expanded configuration.

55. Apparatus for producing a hollow anastomotic connection between a first aperture in a side wall of a graft tissue conduit and a second aperture in a side wall of a body tissue conduit in a patient, comprising:
a first hollow annular connector, wherein the first connector is configured for installation in the first aperture in the side wall of the graft tissue conduit; and
a second hollow annular connector, wherein the second connector is configured for disposition annularly within the second aperture in the side wall of the body tissue conduit, and wherein the first connector is configured for attachment to the second connector such that the second connector is encapsulated by the first connector.
